SEQUENCE LISTING (8 sequences)

SEQ ID NO: 1 - protein, 8 aa, synthetic construct
HLNEDQLR

SEQ ID NO: 2 - protein, 7 aa, synthetic construct
LNEDQLR

SEQ ID NO: 3 - DNA, 21 bp, synthetic construct
  source 1..21: mol_type = other DNA
tgatccttcttgcccctacct

SEQ ID NO: 4 - DNA, 21 bp, synthetic construct
  source 1..21: mol_type = other DNA
ttcttgctgtgagccaccaga

SEQ ID NO: 5 - DNA, 20 bp, synthetic construct
  source 1..20: mol_type = other DNA
agaagaagatcctggcagag

SEQ ID NO: 6 - DNA, 150 bp, synthetic construct
  source 1..150: mol_type = other DNA
agacagagcggaagagtgggaagagacagacagagagagagaagaagaaaatcctggccgagcgaaggaaggcgctggcaatcgatcatctgaatgaagaccaactgaggtggggacagttggttgggtggcccctggcactcttcctga

SEQ ID NO: 7 - DNA, 23 bp, synthetic construct
  source 1..23: mol_type = other DNA
tgggtctttctctcatggtttcc

SEQ ID NO: 8 - DNA, 21 bp, synthetic construct
  source 1..21: mol_type = other DNA
gctcagataagaaaagggcct

(19) United States
(12) Patent Application Publication
(10) Pub. No.: US 2026/0053833 A1
(43) Pub. Date: Feb. 26, 2026

(54) COMPOSITIONS AND METHODS FOR TREATING CARDIOMYOPATHIES

(71) Applicants: THE BOARD OF REGENTS OF THE UNIVERSITY OF TEXAS SYSTEM, Austin, TX (US); THE BOARD OF TRUSTEES OF THE LELAND STANFORD JUNIOR UNIVERSITY, Stanford, CA (US); UNIVERSITY OF CINCINNATI, Cincinnati, OH (US)

(72) Inventors: Hesham A. Sadek, Dallas, TX (US); Ping Wang, Dallas, TX (US); Mahmoud Salama Ahmed, Dallas, TX (US); Joseph Ching-Ming Wu, Dallas, TX (US); Sakthivel Sadayappan, Dallas, TX (US)

(21) Appl. No.: 19/105,737

(22) PCT Filed: Aug. 23, 2023
(86) PCT No.: PCT/US2023/072751

§ 371 Date: Feb. 21, 2025

Related U.S. Application Data
(60) Provisional application No. 63/400,253, filed on Aug. 23, 2022.

(51) Int. Cl. A61K 31/675; A61K 45/06; A61P 9/00

(52) U.S. Cl. CPC A61K 31/675; A61K 45/06; A61P 9/00

(57) ABSTRACT

Compositions and methods for treating a cardiomyopathy, such as dilated cardiomyopathy (DCM) are provided. Embodiments of the present disclosure provide compositions having a bisphosphonate compound, wherein the compound acts as a structure-based corrector therapeutic by targeting a genetic mutation associated with familial DCM.

### CROSS-REFERENCE TO RELATED APPLICATION

This application claims priority to U.S. Provisional Patent Application Ser. No. 63/400,253 filed Aug. 23, 2022, the contents of which is hereby incorporated by reference in its entirety.

### REFERENCE TO SEQUENCE LISTING

This application contains a Sequence Listing that has been submitted in XML format and is hereby incorporated by reference in its entirety. The XML file, created Aug. 22, 2023, is named 106546-768590_UTSD 4054_SequenceListing.xml and is 8,219 bytes in size.

### BACKGROUND

### 1. Field

The present disclosure is directed to compositions and methods of rescuing the dilated cardiomyopathy (DCM) phenotype resulting from a genetic mutation (K210 deletion) of the troponin complex.

### 2. Discussion of Related Art

DCM is a non-ischemic heart muscle disease with structural and functional myocardial abnormalities characterized by left ventricular or biventricular dilation and impaired contraction. Idiopathic and familial disease are the most commonly reported causes of DCM. Genetic mutations in several genes can cause DCM, including single point mutations on genes encoding for sarcomere proteins involved in calcium dynamics. There is no known cure for DCM and the only treatments currently available are invasive, such as surgical repair of the ventricle or implantation of biventricular pacemakers and cardioverter defibrillators. For subjects with advanced DCM, such surgical interventions and be risky and it is unclear whether such surgical treatment always improves long-term outcomes as there is still substantial mortality associated with DCM. As such, there is a need for less invasive therapies, such as pharmaceutical therapeutics, for the treatment of DCM.

### SUMMARY

The present disclosure is based, at least in part, on identification of a bisphosphonate acting as a functional-structure corrector to rescue the DCM phenotype resulting from a genetic mutation (K210 deletion) of the troponin complex. Provided in various embodiments herein are compositions and methods for treating a cardiomyopathy, such as DCM.

In certain embodiments herein, the present disclosure provides methods of improving heart function in a subject in need thereof. In some embodiments, methods disclosed herein may comprise administering at least one bisphosphonate, pharmaceutically acceptable salt thereof, or any hydrate thereof, to a subject in need thereof, wherein the subject in need thereof has or is suspected of having dilated cardiomyopathy (DCM). In some aspects, the at least one bisphosphonate, pharmaceutically acceptable salt thereof, or any hydrate thereof may comprise risedronate.

In some embodiments, methods disclosed herein may improve heart function in a subject that may have or may be suspected of having familial DCM. In some embodiments, methods disclosed herein may improve heart function in a subject that may have or may be suspected of having a deletion mutation at lysine 210 (K210) in a cardiac troponin T gene.

In some embodiments, methods disclosed herein may improve heart function by administering at least one bisphosphonate, pharmaceutically acceptable salt thereof, or any hydrate thereof parenterally. In some aspects, the at least one bisphosphonate, pharmaceutically acceptable salt thereof, or any hydrate thereof may be administered intravenously, subcutaneously, intramuscularly, transdermally, or any combination thereof. In some embodiments, methods disclosed herein may improve heart function by administering at least one bisphosphonate, pharmaceutically acceptable salt thereof, or any hydrate thereof orally.

In some embodiments, methods of administering at least one bisphosphonate, pharmaceutically acceptable salt thereof, or any hydrate thereof may improve heart function in a subject in need thereof compared to an untreated subject with identical disease condition and predicted outcome. In some embodiments, methods of administering at least one bisphosphonate, pharmaceutically acceptable salt thereof, or any hydrate thereof may increase ejection fraction in a subject in need thereof compared to an untreated subject with identical disease condition and predicted outcome. In some embodiments, methods of administering at least one bisphosphonate, pharmaceutically acceptable salt thereof, or any hydrate thereof may decrease cardiac hypertrophy in a subject in need thereof compared to an untreated subject with identical disease condition and predicted outcome.

In certain embodiments herein, the present disclosure provides methods of treating and/or preventing dilated cardiomyopathy (DCM) in a subject. In some embodiments, methods disclosed herein of treating and/or preventing DCM in a subject may comprise administering to a subject having or suspected of having DCM an effective amount of at least one bisphosphonate, pharmaceutically acceptable salt thereof, or any hydrate thereof. In some aspects, the at least one bisphosphonate, pharmaceutically acceptable salt thereof, or any hydrate thereof may comprise risedronate. In some other aspects, an effective amount of the at least one bisphosphonate, pharmaceutically acceptable salt thereof, or any hydrate thereof may comprise an amount that improves at least one characteristic of DCM. In some aspects, the at least one characteristic of DCM may comprise ventricular dilation, systolic dysfunction, or both.

In some embodiments, methods disclosed herein may treat and/or prevent DCM in a subject having or suspected of having familial DCM. In some embodiments, methods disclosed herein may treat and/or prevent DCM in a subject having or suspected of having familial DCM due to a deletion mutation at lysine 210 (K210) in a cardiac troponin T gene.

In some embodiments, methods disclosed herein of treating and/or preventing DCM in a subject may increase fractional shortening, ejection fraction, stroke volume, cardiac output, or any combination thereof in the subject compared to an untreated subject with identical disease condition and predicted outcome.

In some embodiments, methods disclosed herein of treating and/or preventing DCM in a subject may further comprise administering to the subject at least one agent to manage one or more symptoms associated with DCM. In some aspects, agents suitable to manage one or more symptoms associated with DCM may comprise an angiotensin-converting enzyme (ACE) inhibitor, an angiotensin receptor blocker (ARB), a beta-blocker, an aldosterone receptor antagonist, a neural endopeptidase inhibitor, a diuretic, a mineralocorticoid receptor blocker, or any combination thereof. In some aspects, the one or more symptoms associated with DCM may comprise fatigue, dyspnea, edema, heart palpitations, heart murmurs, or any combination thereof.

The foregoing is intended to be illustrative and is not meant in a limiting sense. Many features and subcombinations of the present disclosure may be made and will be readily evident upon a study of the following specification and accompanying drawings comprising a part thereof. These features and subcombinations may be employed without reference to other features and subcombinations.

### BRIEF DESCRIPTION OF THE DRAWINGS

Embodiments of the present disclosure are illustrated by way of example in which like reference numerals indicate similar elements.

FIGS. 1A-1K depict images and graphs illustrating the structure and binding dynamics of a wild type (WT) troponin complex and a troponin complex comprising a K210 deletion in its TnnT2 subunit (a ΔK210 complex, or “ΔK210”). FIG. 1A shows force generation measurement of WT and ΔK210 complex. FIG. 1B shows fluorescence-based measurement of the binding affinity of Ca²⁺ to troponin complex. FIG. 1C shows a cartoon representation of the overall structure of WT and ΔK210 complex. TnnC is shown in green, TnnT is shown in orange and TnnI is shown in purple. Ca²⁺ is shown in pink sphere. This color scheme is consistent for ΔK210 complex in all the following figures unless otherwise specified. FIG. 1D shows a cartoon representation showing superimposition of WT (grey) and ΔK210. FIG. 1E shows surface representation colored by the vacuum electrostatic potential of WT in the same orientation as in FIG. 1C. FIG. 1F shows surface representation colored by the vacuum electrostatic potential of the ΔK210 complex in the same orientation as in FIG. 1C. FIG. 1G shows a cartoon representation highlighting K210 deletion site (in green dash circle) and hinge region of TnnT and TnnI showing superimposition of WT (grey) and ΔK210 (TnnC in green, TnnT in orange and TnnI in purple). Specific residues in the hinge region are shown in stick representation. FIG. 1H shows a cartoon representation showing superimposition of Ca²⁺ binding domains in WT (grey) and ΔK210 (TnnC in green, TnnT in orange and TnnI in purple). FIG. 1I shows a detailed interaction of Ca²⁺ in the activation Ca²⁺ binding pocket for TnnC in ΔK210 complex. Specific residues coordinating the Ca²⁺ are shown in green stick representation. Hydrogen bonds are indicated with black dashed lines. FIG. 1J shows a detailed interaction of Ca²⁺ in the activation Ca²⁺ binding pocket for TnnC in WT complex. Specific residues coordinating the Ca²⁺ are shown in grey stick representation. Hydrogen bonds are indicated with black dashed lines. FIG. 1K shows a cartoon representation showing superimposition of the detailed interaction of Ca²⁺ in the activation Ca²⁺ binding pocket for TnnC in WT complex (grey) and ΔK210 complex (green). Data are mean±s.e.m.; unpaired two-sided t-test. *P<0.05, **P<0.01. ***P<0.001.

FIGS. 2A-2K depict images and graphs illustrating a structure-based approach to identify FDA approved drugs that target a troponin complex comprising a K210 deletion (“ΔK210”). FIG. 2A shows a schematic flow chart for the in silico molecular virtual screening, starting with energy minimized FDA approved small molecules to be followed by semiflexible docking study targeting the hinge region in ΔK210 complex to end up with the top 5 drug candidates. FIG. 2B shows Chemgauss scores for bisphosphonates family members: zoledronic, Risedronic, pamidronic, alendronic, and ibandronic acids. FIG. 2C shows a surface representation colored by the vacuum electrostatic potential of ΔK210 complex. The hinge region is highlighted in black box and risedronate is docked into the hinge region. FIG. 2D shows a cartoon representation of the overall structure of ΔK210 complex in the presence of risedronate. FIG. 2E shows a cartoon representation showing superimposition of WT (grey), ΔK210 (cyan) and ΔK210 complex in the presence of risedronate (purple). FIG. 2F shows a surface representation colored by the vacuum electrostatic potential of ΔK210 complex in the presence of risedronate. FIG. 2G shows a cartoon representation highlighting the hinge region of TnnT and TnnI showing superimposition of WT (grey), ΔK210 (cyan) and ΔK210 complex in the presence of risedronate (purple). Specific residues in the hinge region are shown in stick representation. FIG. 2H shows the detailed interaction of Ca²⁺ in the activation Ca²⁺ binding pocket for TnnC in ΔK210 complex in the presence of risedronate. Specific residues coordinating the Ca²⁺ are shown in green stick representation. Hydrogen bonds are indicated with black dashed lines. FIG. 2I shows a cartoon representation showing superimposition of the detailed interaction of Ca²⁺ in the activation Ca²⁺ binding pocket for TnnC in ΔK210 complex (cyan) and ΔK210 complex in the presence of risedronate (green). FIG. 2J shows a fluorescence-based measurement of the binding affinity of Ca²⁺ to troponin complex at the absence and presence of risedronate. FIG. 2K shows calcium dependent force generation of WT and ΔK210 complex in the presence of 0.5-4 mM risedronate using isolated skinned papillary muscle fibers. The comparison of calcium dependent force generation and calcium sensitivity across control and test experiments was calculated by plotting maximal force generated against the respective pCa. The data was fitted to sigmoidal dose response curve with variable slope (GraphPad Prism 9). Data are mean±s.e.m.; unpaired two-sided t-test. *P<0.05, **P<0.01, ***P<0.001.

FIGS. 3A-3K depict images and graphs illustrating an ex-vivo pharmacological evaluation for K210del mediated dilated cardiomyopathy using iPSC-CM. FIG. 3A shows a schematic for isolation of iPSCs from healthy donors and dilated cardiomyopathy (DCM) patients surrendered ΔK210 TnnT mutations. This was followed by genetically modified DCM derived iPSCs using Crisper/Cas-9. FIG. 3B shows immunofluorescence of TNNT2^{WT} and TNNT2^{K210+/-} stained with DAPI (blue), cTnT (green), and α-actinin (red). FIG. 3C shows pluripotency of iPSCs WT, DCM, and genetically corrected DCM stained with DAPI (blue), SOX2 (green), NANOG (red), and OCT3/4 (purple). FIG. 3D shows trilineage differentiation for the endoderm of iPSCs for WT, DCM, and genetically corrected DCM stained with DAPI (blue), SOX17 (green), and FOXA2 (red). FIG. 3E shows trilineage differentiation for the mesoderm of iPSCs for WT, DCM, and genetically corrected DCM stained with DAPI (blue), Brachyury (green), and TBX6 (red). FIG. 3F shows trilineage differentiation for the ectoderm of iPSCs for WT, DCM, and genetically corrected DCM stained with DAPI (blue), OTX2 (green), and PAX6 (red). FIG. 3G shows morphology of iPSCs for WT, DCM, and genetically corrected DCM. FIG. 3H shows contraction velocity for WT, DCM, and genetically corrected DCM iPSCs. FIG. 3I shows contraction velocity for DCM and genetically corrected DCM ipSCs from Day 0 until Day 7. FIG. 3J shows contraction velocity for genetically corrected DCM iPSCs treated with zoledronic acid, pamidronate, and risedronate with no difference at two dose levels. FIG. 3K shows contraction velocity for DCM iPSCs treated with zoledronic acid, pamidronate, and risedronate at two dose levels, where risedronate showed significant increase the contraction velocity of DCM derived iPSCs. Data are mean±s.e.m.; unpaired two-sided t-test. *P<0.05, **P<0.01, ***P<0.001.

FIGS. 4A-4N depict images and graphs illustrating an in vivo pharmacological evaluation of risedronate a DCM mouse model. FIG. 4A shows a DNA sequencing chromatogram of TnnT^{K210+/-} BALB/C mice from genomic DNA PCR products. FIG. 4B shows a schematic for risedronate administration to 5-6 weeks old TnnT^{K210}+/− BALB/C mice at 150 μg/kg/day for 15 weeks while monitoring ejection fraction using echocardiography and magnetic resonance imaging (MRI). FIG. 4C shows a serial echocardiography assessment of LVEF showing elevated LVEF for risedronate-treated TNNT2^{K210+/-} (150 μg/kg/day), compared with controls. FIG. 4D shows Masson's trichrome staining of hearts, 15-weeks post-risedronate administration (150 μg/kg/day), showing non-significant change in the interstitial fibrosis, compared with control-treated TNNT2^{K210+/-} mice. FIGS. 4E-4G show representative images of MRI showing elevated LVEF and lower circumferential strain for risedronate-treated TNNT2^{K210+/-} (150 μg/kg/day), compared with controls. FIGS. 4H-4I show WGA staining and CSA quantification showing a significant decrease in cardiomyocyte cell size for risedronate-treated TNNT2^{K210+/-} (150 μg/kg/day), compared with controls. FIGS. 4J-4K show a representative echocardiography images and serial echocardiography for cross-over study for risedronate-treated TNNT2^{K210+/-} and control-treated TNNT2^{K210+/-} mice. FIG. 4L shows a schematic for risedronate administration to 5-6 weeks old TnnT^{K210}+/− BALB/C mice at 75 μg/kg/day for 11 weeks while monitoring ejection fraction using echocardiography. FIG. 4M shows representative echocardiography images for risedronate-treated TNNT2^{K210+/-} (75 μg/kg/day) and control-treated TNNT2^{K210+/-} mice. FIG. 4N shows a serial echocardiography assessment of LVEF showing elevated LVEF for risedronate-treated TNNT2^{K210+/-} (75 μg/kg/day), compared with control-treated TNNT2^{K210+/-} mice. Data are mean±s.e.m.; unpaired two-sided t-test. *P<0.05, **P<0.01, ***P<0.001.

FIGS. 5A-5D depict images and graphs illustrating a wild type troponin complex assembled in vitro by co-expressing three components of troponin complex (TnnI, TnnT2 and TnnC) together. FIG. 5A shows a size-exclusion chromatography (Superdex 200) of the WT complex. The Y axis shows the absorbance at 280 nm and the X axis shows the elution volume in ml. Peak fractions were analyzed by SDS-PAGE and visualized with Stain-Free dye (Biorad).

FIG. 5B shows a cartoon representation of the overall structure of ΔK210 complex colored by b factor (left) and a cartoon representation showing superimposition of two protomers in the asymmetric unit of ΔK210 complex colored by b factor (right in the red box). FIG. 5C shows a cartoon representation of the overall structure of WT complex colored by b factor (left) and a cartoon representation showing superimposition of two protomers in the asymmetric unit of WT complex colored by b factor (right in the red box). FIG. 5D shows a cartoon representation of the overall structure of p WT complex (PDB: 1J1D) colored by b factor (left) and a cartoon representation showing superimposition of two protomers in the asymmetric unit of published WT complex colored by b factor (right in the red box).

FIGS. 6A-6E depict images illustrating structure comparison between the exemplary WT structure prepared herein and the published WT. FIG. 6A shows a cartoon representation showing superimposition of WT (TnnC in green, TnnT in orange and TnnI in purple) and published WT complex (PDB: 1J1D). FIG. 6B shows a surface representation colored by the vacuum electrostatic potential of the published WT complex. FIG. 6C highlights K210 site and hinge region of TnnT and TnnI showing superimposition of WT (TnnC in green. TnnT in orange and TnnI in purple) and published WT (grey). FIG. 6D shows a cartoon representation showing superimposition of TnnC in WT and published WT (grey). FIG. 6E shows a cartoon representation showing superimposition of the detailed interaction of Ca²⁺ in the activation Ca²⁺ binding pocket for TnnC in WT (green) and published WT (grey).

FIGS. 7A-7C depict images illustrating structure comparison between the exemplary WT structure and ΔK210. FIG. 7A shows a simulated annealing omit map of Ca²⁺ coordination in the activation Ca²⁺ binding pocket for TnnC in ΔK210 complex. TnnC is shown in green, TnnT is shown in orange and TnnI is shown in purple. Ca²⁺ is shown in pink sphere. FIG. 7B shows a simulated annealing omit map of Ca²⁺ coordination in the activation Ca²⁺ binding pocket for TnnC in WT complex. FIG. 7C shows a cartoon representation showing superimposition of the structural Ca²⁺ binding domain of TnnC in WT (grey) and ΔK210 (TnnC in green, TnnT in orange and TnnI in purple).

FIGS. 8A-8H depict images illustrating structures of ΔK210-risedronate. FIG. 8A shows RMSD, RMSF, Radius of gyration, and number of hydrogen bonds are the main parameters for molecular dynamic simulations over 20 ns for mutated (K210 del) alone and with risedronate. FIG. 8B shows a cartoon representation of the overall structure of ΔK210 complex in the presence of risedronate acid colored by b factor (left) and a cartoon representation showing superimposition of two protomers in the asymmetric unit of ΔK210 complex in the presence of risedronate acid colored by b factor (right in the red box). FIG. 8C shows a cartoon representation showing superimposition of ΔK210 complex (cyan) and ΔK210 complex in the presence of risedronate (TnnC in green, TnnT in orange and TnnI in purple). FIG. 8D shows a cartoon representation showing superimposition of WT complex (grey) and ΔK210 complex in the presence of risedronate (TnnC in green, TnnT in orange and TnnI in purple). FIG. 8E shows a cartoon representation showing superimposition of TnnC in ΔK210 complex in the presence of risedronate (purple) and ΔK210 complex (cyan). FIG. 8F shows simulated annealing omit map of Ca²⁺ coordination in the activation Ca²⁺ binding pocket for TnnC in ΔK210 complex in the presence of risedronate. TnnC is shown in green, TnnT is shown in orange and TnnI is shown in purple. Ca²⁺ is shown in pink sphere. FIG. 8G shows a cartoon representation showing superimposition of the detailed interaction of Ca²⁺ in the activation Ca²⁺ binding pocket for TnnC in WT complex (grey), ΔK210 complex (cyan) and ΔK210 complex in the presence of risedronate acid (purple). Specific residues coordinating the Ca²⁺ are shown in stick representation. FIG. 8H shows the detailed interaction of Ca²⁺ in the activation Ca²⁺ binding pocket for TnnC in ΔK210 complex in the presence of different bisphosphonate family members (Elandroante in purple, Neridronate in cyan, Ibandronate in yellow and Pamidronate in grey). Specific residues coordinating the Ca²⁺ are shown in stick representation. Hydrogen bonds are indicated with black dashed lines.

FIGS. 9A-9B depict images illustrating Sanger sequencing for WT, DCM, and genetically corrected DCM iPSCs (FIG. 9A) and a karyotype for WT, DCM, and genetically corrected DCM iPSCs (FIG. 9B).

FIGS. 10A-10D depict images and graphs illustrating an in vivo pharmacological evaluation of risedronate a DCM mouse model. FIG. 10A shows heart/body weight for TNNT2^{K210+/-}, risedronate-treated TNNT2^{K210+/-} (150 μg/kg/day), and TNNT2^{WT}. FIG. 10B shows heart/body weight for risedronate-treated TNNT2^{K210+/-} (75 μg/kg/day) and control-treated TNNT2^{K210+/-} mice. FIGS. 10C-10D show Masson's trichrome staining of hearts, 6-weeks post-risedronate administration (75 μg/kg/day) in TNNT2^{K210+/-} mice, showing non-significant change in the interstitial fibrosis, compared with control-treated TNNT2^{K210+/-} hearts.

The drawing figures do not limit the present disclosure to the specific embodiments disclosed and described herein. The drawings are not necessarily to scale, emphasis instead being placed on clearly illustrating principles of certain embodiments of the present disclosure.

### DETAILED DESCRIPTION

The following detailed description references the accompanying drawings that illustrate various embodiments of the present disclosure. The drawings and description are intended to describe aspects and embodiments of the present disclosure in sufficient detail to enable those skilled in the art to practice the present disclosure. Other components can be utilized and changes can be made without departing from the scope of the present disclosure. The following description is, therefore, not to be taken in a limiting sense.

Troponin complex is a component of cardiac muscle thin filaments. The complex consists of three subunits—Troponin I (TnnI), Troponin T (TnnT) and Troponin C (TnnC). The subunits combine to form the complex which plays a crucial role in cardiac muscle activity, connecting changes in intracellular calcium (Ca²⁺) concentration with generation of contraction. Single amino acid deletions or interchanges can result in significant alterations for the protein structure and/or function, leading to diseases associated with misfolded proteins commonly referred to as “proteinopathies.” Several genetic mutations in Troponin T are associated with different forms of cardiomyopathies, including dilated cardiomyopathy (DCM) or hypertrophic cardiomyopathy (HCM). Calcium desensitization of myofilaments is indicated as a primary mechanism for the pathogenesis of DCM resulting from the deletion of lysine 210 (ΔK210) in cardiac TnnT. The K210 deletion not only reduces contractility in mutant cardiomyocytes but also causes cellular hypertrophy and impairs cardiomyocytes' ability to adapt to changes in substrate stiffness. However, the structure consequence of K210 and how it results in DCM is still elusive. The present disclosure is based, at least in part, on identification of a bisphosphonate acting as a functional-structure corrector to rescue the DCM phenotype resulting from a genetic mutation (K210 deletion) of the troponin complex.

Provided herein are compositions and methods for treating DCM. In certain embodiments, a bisphosphonate (e.g., risedronate) can be provided to improve at least one characteristic of DCM, such as ventricular dilation, systolic dysfunction, or both.

### I. Terminology

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises.” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results. The term “therapeutically effective amount,” as used herein, means an amount of a compound or combination of compounds that ameliorates, attenuates, or eliminates one or more symptoms of DCM or prevents or delays the onset of one or more symptoms of DCM as defined herein.

As used herein, “individual,” “subject,” “host,” and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject can be a human. In other embodiments, the subject can be a human in need of treating a cardiomyopathy (e.g., DCM).

### II. Compositions

The present disclosure provides for compositions for treating a cardiomyopathy. The term “cardiomyopathy” as used herein, means the deterioration of the function of the myocardium (i.e., the actual heart muscle) for any reason. People with cardiomyopathy are often at risk of arrhythmia and/or sudden cardiac death. Cardiomyopathies can generally be categorized into extrinsic cardiomyopathies and intrinsic cardiomyopathies. Extrinsic cardiomyopathies are cardiac disorders where the primary pathology is outside the myocardium itself. Most cardiomyopathies are extrinsic as the underlying myocardial injury is due to extrinsic factors such as ischemia. Examples of extrinsic cardiomyopathies include ischemic cardiomyopathy and cardiomyopathy due to systemic diseases. Ischemic cardiomyopathy is a weakness in the muscle of the heart due to inadequate oxygen delivery to the myocardium with coronary artery disease being the most common cause. Intrinsic cardiomyopathies are cardiac disorders where weakness in the muscle of the heart is not due to an identifiable external cause. Intrinsic cardiomyopathies—also known as “familial cardiomyopathies”—usually result from one or more genetic mutations and include dilated cardiomyopathy (DCM), hypertrophic cardiomyopathy (HCM or HOCM), arrhythmogenic right ventricular cardiomyopathy (ARVC), and restrictive cardiomyopathy (RCM). Embodiments of the present disclosure provide compositions for treating DCM. In certain embodiments, compositions for treating DCM may comprise at least one bisphosphonate, pharmaceutically acceptable salt thereof, or any hydrate thereof. In other certain embodiments, compositions for treating DCM may be pharmaceutical compositions comprising at least one bisphosphonate, pharmaceutically acceptable salt thereof, or any hydrate thereof and at least one suitable carrier or excipient.

### (a) Bisphosphonates

In certain embodiments, compositions for treating DCM may comprise at least one bisphosphonate, pharmaceutically acceptable salt thereof, or any hydrate thereof. As used herein, the term “bisphosphonate” refers to any compound which is an analog of endogenous pyrophosphate whereby the central oxygen is replaced by carbon. Bisphosphonates can include aminobisphosphonates. Bisphosphonates can also include, but are not limited to, the following compounds: zoledronic acid, risedronate, alendronate, cimadronate, clodronate, tiludronate, etidronate, ibandronate, piridronate, or pamidronate and functional analogues thereof. In some embodiments, bisphosphonates for use in the compositions disclosed herein can include acids, salts, esters, hydrates, polymorphs, hemihydrates, solvates, and derivatives thereof. Non-limiting examples of salts useful herein include those selected from the group consisting of alkali metal, alkaline metal, ammonium, and mono-, di-, tri-, or tetra-C1-C30-alkyl-substituted ammonium. Preferred salts are those selected from the group consisting of sodium, potassium, and ammonium salts. In preferred embodiments, compositions for treating DCM may comprise at least one bisphosphonate, pharmaceutically acceptable salt thereof, or any hydrate thereof wherein the bisphosphonate is risedronate. Risedronate, also known as NE-58095 or risedronic acid, is an aminobisphosphonate. Its chemical name is 2-hydroxyethylidene-2-(3-pyridinyl)-1,1bisphosphonate disodium.

### (b) Pharmaceutical Formulations and Treatment Regimens

In certain embodiments, any one or more active agents disclosed herein (e.g., a bisphosphonate, such as risedronate) may be provided per se or as part of a pharmaceutical composition, where the active agent(s) can be mixed with suitable carriers or excipients. As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

### (i) Pharmaceutically Acceptable Carriers and Excipients

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

In certain embodiments, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, antioxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “REMINGTON'S PHARMACEUTICAL SCIENCES,” Mack Publishing Co., Easton, Pa., latest edition, which is incorporated herein by reference.

In certain embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In certain embodiments, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition.

In certain embodiments, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethylcellulose (CMC), silicon dioxide, polyvinylpyrrolidone (PVP: povidone), Splenda® (dextrose, maltodextrin and sucralose), or combinations thereof. In some embodiments, suitable thickening agent may be carboxymethyl-cellulose.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the present disclosure. In s aspects, additives will be biocompatible, and will not be harsh, abrasive, or allergenic.

In certain embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In certain embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In certain embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In certain embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In a preferred embodiment, compositions disclosed herein may have a pH greater than about 6.8.

In certain embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In certain embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In certain embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In certain embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L.

### (ii) Dosage Formulations

In certain embodiments, the present disclosure provides compositions formulated for one or more routes of administration. Suitable routes of administration may, for example, include oral, rectal, transmucosal, transnasal, intestinal, and/or parenteral delivery. In some embodiments, compositions herein formulated can be formulated for parenteral delivery. In some embodiments, compositions herein formulated can be formulated intramuscular, subcutaneous, intramedullary, intravenous, intraperitoneal, and/or intranasal injections.

In certain embodiments, one may administer a composition herein in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or a combination thereof. In some embodiments, a pharmaceutical composition disclosed herein can administered to subject as disclosed herein. In some embodiments, a pharmaceutical composition disclosed herein can administered to human patient. In some embodiments, a pharmaceutical composition disclosed herein can administered to a human patient via at least two administration routes. In some embodiments, the combination of administration routes by be intracerebroventricular injection and intravenous injection; intrathecal injection and intravenous injection; intra-cisterna magna injection and intravenous injection; and/or intra-parenchymal injection and intravenous injection.

In certain embodiments, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain embodiments, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain embodiments, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some embodiments, compositions herein may be suspensions, solutions or emulsions in oily or aqueous vehicles, and/or may contain formulatory agents such as suspending, stabilizing and/or dispersing agents.

In certain embodiments, pharmaceutical compositions herein formulated for parenteral administration may include aqueous solutions of the active preparation (e.g., a bisphosphonate, such as risedronate) in water-soluble form. In some embodiments, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some embodiments, compositions herein comprising aqueous injection suspensions may contain substances which increase the viscosity of the suspension, such as sodium carboxymethyl cellulose, sorbitol, and/or dextran. In some embodiments, compositions herein comprising a suspension may also contain one or more suitable stabilizers and/or agents which increase the solubility of the active ingredients (e.g., a bisphosphonate, such as risedronate) to allow for the preparation of highly concentrated solutions.

In some embodiments, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some embodiments, a therapeutically effective amount means an amount of active ingredients (e.g., a bisphosphonate, such as risedronate effective to prevent, slow, alleviate or ameliorate symptoms of a disorder (e.g., DCM) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

In some embodiments, toxicity and therapeutic efficacy of the active ingredients disclosed herein can be determined by standard pharmaceutical procedures in vitro, in cell cultures or experimental animals. In some embodiments, data obtained from these in vitro and cell culture assays and animal studies can be used in formulating a range of dosage for use in a human subject. In some embodiments, a dosage for use herein may vary depending upon the dosage form employed and the route of administration utilized. The exact formulation, route of administration and dosage can be chosen by the individual physician in view of the patient's condition. (See e.g., Fingl, et al., 1975, in “THE PHARMACOLOGICAL BASIS OF THERAPEUTICS”).

In certain embodiments, dosage amounts and/or dosing intervals may be adjusted individually to brain or blood levels of the active ingredient that are sufficient to induce or suppress the biological effect (minimal effective concentration, MEC). In some embodiments, the MEC for an active ingredient (e.g., a bisphosphonate, such as risedronate) may vary for each preparation but can be estimated from in vitro data. In some embodiments, dosages necessary to achieve the MEC herein may depend on individual characteristics and route of administration. Detection assays can be used to determine plasma concentrations.

In certain embodiments, depending on the severity and responsiveness of the condition to be treated, dosing with compositions herein can be of a single or a plurality of administrations, with course of treatment lasting from several days to several weeks or until cure is affected or diminution of the disease state is achieved.

In certain embodiments, amounts of a composition herein to be administered will be dependent on the subject being treated, the severity of the affliction, the manner of administration, the judgment of the prescribing physician, and the like. In some embodiments, effective doses may be extrapolated from dose-responsive curves derived from in vitro or in vivo test systems.

In certain embodiments, the amount of bisphosphonate (e.g., risedronate) contained in the dosage forms of the present disclosure will depend on the particular bisphosphonate form selected and the continuous dosing schedule upon which the bisphosphonate is dosed to the subject. Continuous dosing schedules of daily, weekly, twice monthly, three times per month, and once monthly are non-limiting examples of dosing regimens suitable for use with the dosage forms of the present disclosure. The terms “three times per month” or “thrice monthly” mean that an oral dosage form is administered thrice, i.e., three times, during a monthly calendar period. In a thrice monthly schedule, the dosage forms disclosed herein may be administered on three consecutive days, or once about every nine to eleven days. The terms “twice per month” or “twice monthly” mean that an oral dosage form is administered twice, i.e., two times, during a monthly calendar period. In a twice monthly regimen, the dosage forms disclosed herein may be administered on consecutive days or once about every fourteen to sixteen days. The terms “monthly” or “once monthly” mean that a dosage form disclosed herein is administered once, i.e., one time during a monthly calendar period, that is, about every 28 to 31 days.

Mixed nomenclature is currently in use by those of ordinary skill in the art, for example reference to a specific weight or percentage of a bisphosphonate active ingredient is on an anhydrous monosodium salt basis for risedronate. In some embodiments, the phrase “about 35 mg of risedronate, pharmaceutically acceptable salts thereof, and mixtures thereof, on an anhydrous monosodium salt basis” means that the amount of the risedronate compound selected is calculated based on about 35 mg of anhydrous risedronate monosodium salt.

In some embodiments, dosage forms disclosed herein may be oral dosage forms. In some embodiments, oral dosage forms disclosed herein may comprise risedronate. In some embodiments, oral dosage forms of the present disclosure may contain from about 1 mg to about 250 mg of risedronate on a risedronate anhydrous monosodium salt basis. A daily oral dosage form as embodied herein may contain from about 1 mg to about 10 mg risedronate on a risedronate anhydrous monosodium salt basis. A weekly oral dosage form as embodied herein may contain from about 10 to about 70 mg risedronate on a risedronate anhydrous monosodium salt basis, preferably from 15 to about 55 mg risedronate, more preferably from about 35 mg to about 50 mg risedronate. A twice monthly oral dosage form as embodied herein may contain from about 20 to about 120 mg risedronate, preferably about 75 mg to about 90 mg risedronate on a risedronate anhydrous monosodium salt basis. In some embodiments, an oral dosage form disclosed herein that is administered three times per month contains from about 15 to about 90 mg risedronate, preferably about 50 mg to about 75 mg risedronate, on a risedronate anhydrous monosodium salt basis. A monthly oral dosage form as embodied herein may contain from about 50 to about 280 mg risedronate, preferably from about 100 to about 250 mg risedronate, and more preferably about 150 to about 200 mg risedronate on a risedronate anhydrous monosodium salt basis. In some embodiments, an oral dosage form disclosed herein may contain about 100% of the effective amount of the risedronate as equivalent non-chelating agent containing, non-delayed, immediate released risedronate tablets. In some embodiments, risedronate may be administered from about 1 mg to about 250 mg orally a day depending on the subject's clinical status. In some embodiments, risedronate may be administered from about 1 mg, about 5 mg, about 25 mg, about 50 mg, about 100 mg, about 150 mg, about 200 mg, or about 250 mg orally a day depending on the subject's clinical status.

### III. Methods of Use

The present disclosure provides for methods of treating, attenuating, and preventing DCM in a subject in need thereof. The present disclosure also provides for methods of improving heart function, increasing ejection fraction, decreasing cardiac hypertrophy, or any combination thereof compared to an untreated subject with identical disease condition and predicted outcome. In certain embodiments, a method for treating, attenuating, or preventing DCM in a subject can include administering to a subject, including a human subject having or suspected of having familial DCM resulting from a deletion mutation at lysine 210 (K210) in a cardiac troponin T gene, an effective amount of a composition as disclosed herein.

A suitable subject includes a human, a livestock animal, a companion animal, a lab animal, or a zoological animal. In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In certain embodiments, a subject in need may have been diagnosed with at least one heart disease. In some embodiments, the subject may have or be suspected of having a cardiomyopathy. In some embodiments, the subject may have or be suspected of having DCM. A subject having DCM can be identified by routine medical examination, e.g., laboratory tests, EKG, ECG, echocardiogram, stress tests, MRI, coronary angioplasty, myocardial bioposy, organ functional tests, CT scans, or ultrasounds. In some embodiments, the subject to be treated by the methods described herein may be a human patient who has shown one or more symptoms of DCM. Non-limiting symptoms of DCM include fatigue, dyspnea, edema, heart palpitations, heart murmurs, and the like.

In some embodiments, the subject to be treated by the methods described herein may have or be suspected of having familial DCM. In some aspects, a subject having or suspected of having familial DCM may have at least one genetic mutation in a protein of the cardiac troponin complex. In some other aspects, a subject having or suspected of having familial DCM may have at least one genetic mutation in Troponin I (TnnI), Troponin T (TnnT), Troponin C (TnnC), or any combination thereof. In still some other aspects, a subject having or suspected of having familial DCM may have a deletion mutation at lysine 210 (K210) in a cardiac troponin T gene. In some embodiments, a subject in need of any of the methods herein may have a genetic mutation associated with DCM with no disease manifestations at the time of the treatment.

In certain embodiments, methods disclosed herein may improve heart function in a subject in need thereof compared to an untreated subject with identical disease condition and predicted outcome. In some embodiments, methods disclosed herein may improve heart function in a subject in need thereof from about 1% improvement to about 100% improvement compared to an untreated subject with identical disease condition and predicted outcome. In some embodiments, methods disclosed herein may improve heart function in a subject in need thereof from about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% improvement compared to an untreated subject with identical disease condition and predicted outcome.

In certain embodiments, methods disclosed herein may reduce cardiac fibrosis in a subject in need thereof compared to an untreated subject with identical disease condition and predicted outcome. In some embodiments, methods disclosed herein may reduce cardiac fibrosis in a subject in need thereof from about 1% to about 100% compared to an untreated subject with identical disease condition and predicted outcome. In some embodiments, methods disclosed herein may reduce cardiac fibrosis in a subject in need thereof from about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to an untreated subject with identical disease condition and predicted outcome.

In certain embodiments, methods disclosed herein may reverse and/or prevent cardiac hypertrophy in a subject in need thereof compared to an untreated subject with identical disease condition and predicted outcome. In some embodiments, methods disclosed herein may reverse and/or prevent cardiac hypertrophy in a subject in need thereof from about 1% to about 100% compared to an untreated subject with identical disease condition and predicted outcome. In some embodiments, methods disclosed herein may reverse and/or prevent cardiac hypertrophy in a subject in need thereof from about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to an untreated subject with identical disease condition and predicted outcome.

In certain embodiments, methods disclosed herein may increase fractional shortening in a subject in need thereof compared to an untreated subject with identical disease condition and predicted outcome. In some embodiments, methods disclosed herein may increase fractional shortening in a subject in need thereof from about 1% to about 100% compared to an untreated subject with identical disease condition and predicted outcome. In some embodiments, methods disclosed herein may increase fractional shortening in a subject in need thereof from about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to an untreated subject with identical disease condition and predicted outcome.

In certain embodiments, methods disclosed herein may increase ejection fraction in a subject in need thereof compared to an untreated subject with identical disease condition and predicted outcome. In some embodiments, methods disclosed herein may increase ejection fraction in a subject in need thereof from about 1% to about 100% compared to an untreated subject with identical disease condition and predicted outcome. In some embodiments, methods disclosed herein may increase ejection fraction in a subject in need thereof from about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to an untreated subject with identical disease condition and predicted outcome.

In certain embodiments, methods disclosed herein may increase stroke volume in a subject in need thereof compared to an untreated subject with identical disease condition and predicted outcome. In some embodiments, methods disclosed herein may increase stroke volume in a subject in need thereof from about 1% to about 100% compared to an untreated subject with identical disease condition and predicted outcome. In some embodiments, methods disclosed herein may increase stroke volume in a subject in need thereof from about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to an untreated subject with identical disease condition and predicted outcome.

In certain embodiments, methods disclosed herein may increase cardiac output in a subject in need thereof compared to an untreated subject with identical disease condition and predicted outcome. In some embodiments, methods disclosed herein may increase cardiac output in a subject in need thereof from about 1% to about 100% compared to an untreated subject with identical disease condition and predicted outcome. In some embodiments, methods disclosed herein may increase cardiac output in a subject in need thereof from about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to an untreated subject with identical disease condition and predicted outcome.

Conventional methods, known to those of ordinary skill in the art of medicine, can be used to administer the compositions disclosed herein to a subject, depending upon the type of disease to be treated or the site of the disease. In some embodiments, compositions herein can be administered to a subject by intravenous infusion, by subcutaneous administration, by inhalation, by intranasal administration or other mode of administration. In some embodiments, compositions herein can be administered to a subject orally.

In some embodiments, any of the methods disclosed herein can further include monitoring occurrence of one or more adverse effects in the subject. Exemplary adverse effects include, but are not limited to, hepatic impairment, hematologic toxicity, neurologic toxicity, cutaneous toxicity, gastrointestinal toxicity, or a combination thereof. When one or more adverse effects are observed, the method disclosed herein can further include reducing or increasing the dose of one or more of the disclosed active agents (e.g., risedronate) depending on the adverse effect or effects in the subject. For example, when a moderate to severe hepatic impairment is observed in a subject after treatment, the amount of a composition disclosed herein can be reduced in concentration, frequency of dosing, or a combination thereof.

In some embodiments, methods herein of treating DCM as disclosed herein can further include treating a subject with at least one additional therapeutic regimen for heart failure (HF) in general and/or DCM. Non-limiting examples include medications, surgery, enzyme replacement therapy, hematopoietic stem cell (HSC) transplantation, substrate reduction molecule therapy, chaperone therapy, adeno-associated virus gene therapy, HSC-mediated lentiviral vector gene therapy, or the combined therapy disclosed herein. Examples of medications for HR/DCM can include, but are not limited to, angiotensin-converting enzyme (ACE) inhibitors (i.e., enalapril, Lisinopril, captopril), angiotensin II receptor blockers (i.e., losartan, valsartan, candesartan), beta blockers (i.e., carvedilol, metoprolol, bisoprolol), diuretics (i.e., furosemide, thiazide, spironolactone), inotropes, digoxin, aldosterone receptor antagonists, neural endopeptidase inhibitors, mineralocorticoid receptor blockers, and the like. Examples of surgery for the treatment of for HR/DCM can include, but are not limited to, coronary artery bypass surgery, heart valve repair or replacement, annuloplasty, implantable cardioverter-defibrillators, cardiac resynchronization therapy (CRT), biventricular pacing, ventricular assist devices (VADs), heart transplants, and the like. In some embodiments, a subject treated with any of the methods herein can have completed an additional therapeutic regimen, be receiving an additional therapeutic regimen, or can receive an additional therapeutic regimen following treatment according to the methods herein.

### IV. Kits

The present disclosure provides kits for use in treating or alleviating a cardiomyopathy, such as DCM described herein. Such kits can include one or more containers including one or more disclosed active agents (e.g., risedronate). In some embodiments, kits can include one or more containers including one or more one or more disclosed active agents (e.g., a bisphosphonate, such as risedronate).

In some embodiments, the kits herein can include instructions for use in accordance with any of the methods described herein. The included instructions can have a description of administration of the one or more disclosed active agents (e.g., a bisphosphonate, such as risedronate) to treat, delay the onset, or alleviate a target disease (e.g., DCM) as those described herein, or a combination thereof. In some embodiments, the kit can further include a description of selecting an individual suitable for treatment based on identifying whether that individual has the target disease, e.g., applying a diagnostic method as described herein. In still other embodiments, the instructions can have a description of administering any one of the compositions described herein to an individual at risk of the target disease.

In some embodiments, kit instructions relating to the use of one or more disclosed active agents (e.g., a bisphosphonate, such as risedronate) can generally include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease and/or symptom thereof (e.g., DCM). In some embodiments, instructions are provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container can be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle).

In some embodiments, kits herein can optionally provide additional components such as buffers and interpretive information. Normally, the kit comprises a container and a label or package insert(s) on or associated with the container. In some embodiments, the disclosure provides articles of manufacture comprising contents of the kits described above.

Having described several embodiments, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Introduction to Examples 1-7

Dilated Cardiomyopathy (DCM) is the leading cause of sudden cardiac death, and it is the most common indication of heart transplant. DCM is characterized by left ventricular chamber enlargement and diminished systolic performance. Single point mutations on sarcomere proteins that are associated with decreased calcium (Ca²⁺) sensitivity can cause DCM. As an example, mutations in one or more of the subunits that make up a troponin complex (i.e., Troponin I (TnnI), Troponin T (TnnT) and Troponin C (TnnC)) are associated with hypertrophic, dilated and restrictive cardiomyopathies. A deletion mutation Lysine 210 (K210del) in TnnT2 is a DCM-associated mutation that decreases Ca²⁺ binding affinity, reduces contractility in mutant cardiomyocytes, causes cellular hypertrophy, and impairs cardiomyocytes' ability to adapt to changes in substrate stiffness. Exemplary methods provided in the present disclosure are toward discovery of structure-based corrector therapeutics targeting Tnnt K210del as a treatment of DCM.

### Example 1. Calcium and Magnesium Binding Dynamics of WT and ΔK210

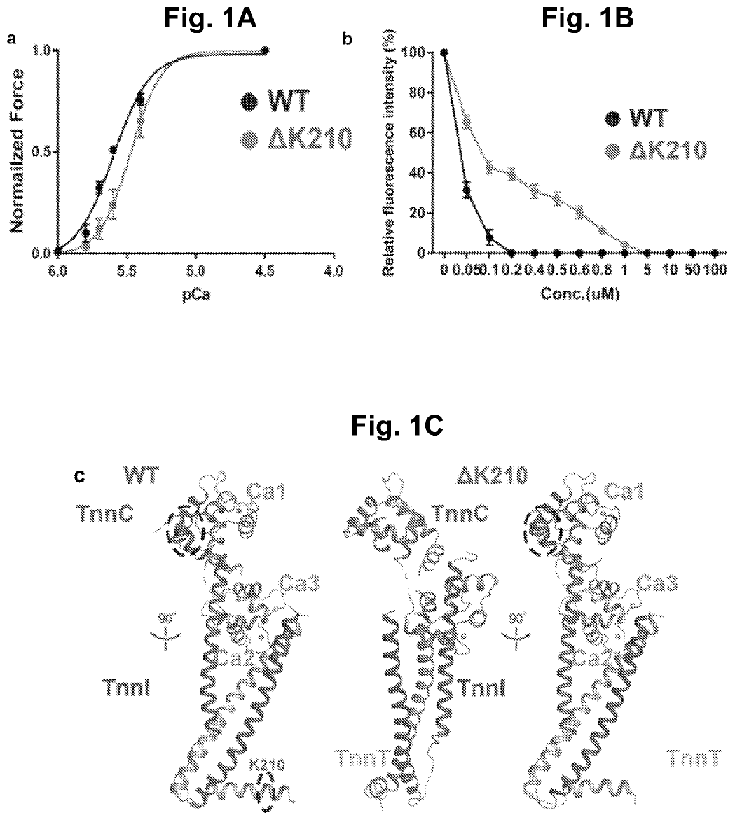

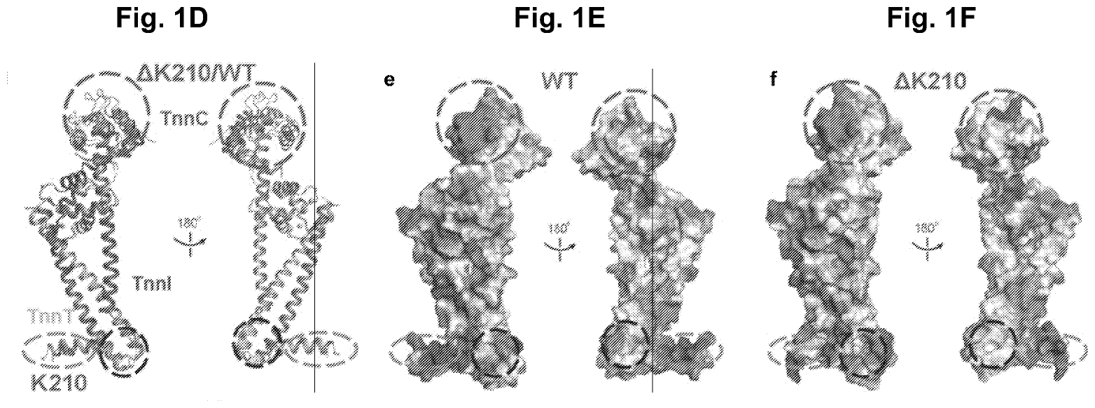

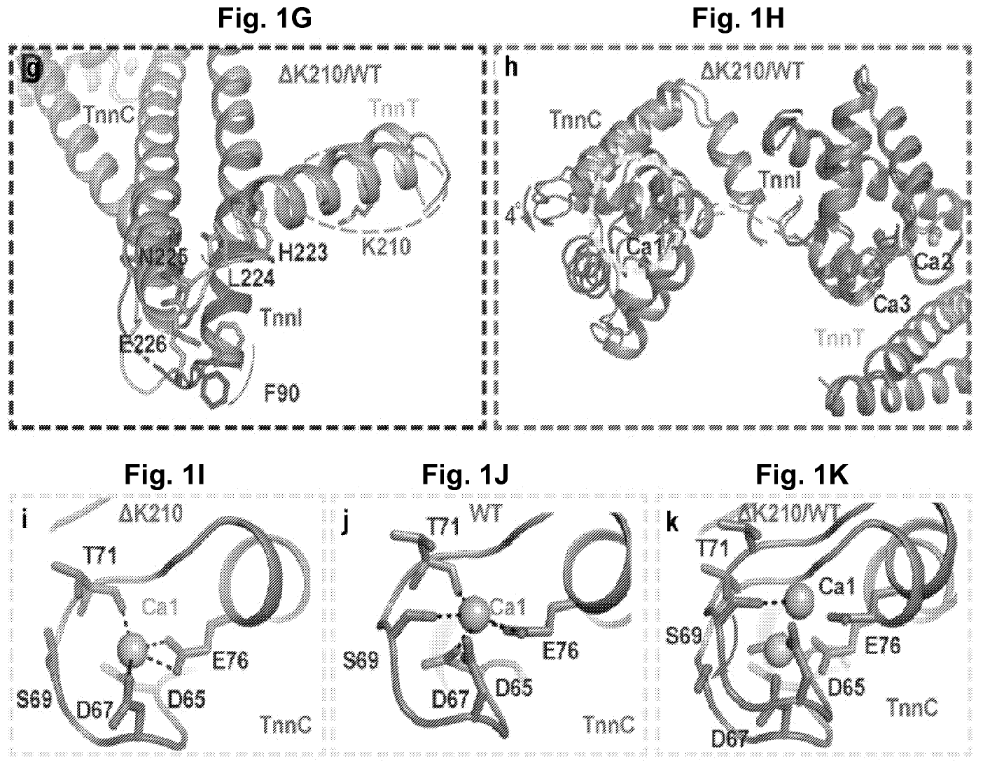

To assess if decreased calcium sensitivity for the DCM-associated mutation K210del was due to impaired Ca²⁺ stimulus response at the sarcomere level and troponin complex level, the force generation of a skinned papillary muscle harvested from wild type mice and K210 deletion heterozygous (ΔK210het) mice were measured. The muscle from ΔK210het mice generated less maximal force at lower concentrations of Ca²⁺ but was able to catch up with that of the wild type mouse muscle preparation at a high concentration of Ca²⁺. Also, the right-shift of the curve for ΔK210het compared with wild type indicated that calcium sensitivity was decreased upon K210 deletion (FIG. 1A).

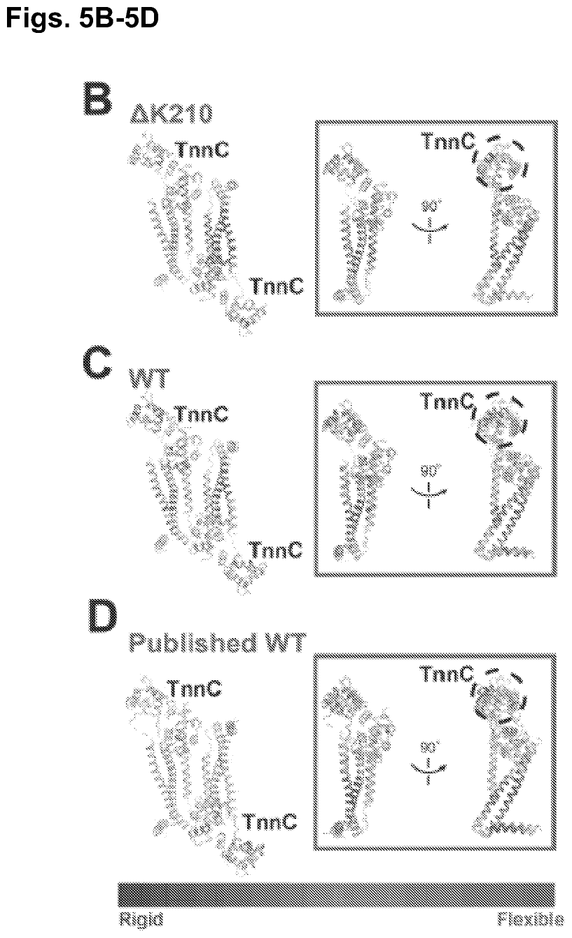

To investigate the effect of the K210 deletion on Ca²⁺ binding/Ca²⁺ exchange at the activation domain of TnnC in vitro, a F27W reporter was utilized the to monitor the fluorescence change during the Ca²⁺ titration. A wild type troponin complex (“WT” hereafter) and K210 deletion on TnnT2 containing troponin complex (“ΔK210” hereafter) were first assembled in vitro by co-expressing three components of troponin complex (TnnI, TnnT2 and TnnC) together (FIG. 5A). Then F27W was introduced to TnnC in the context of WT and ΔK210 to generate WT-F27W and ΔK210-F27W. The fluorescence change upon Ca²⁺ titration indicated that the response to Ca²⁺ stimulus was different between WT-F27W and ΔK210-F27W (FIG. 1B). Collectively, results revealed that the K210 deletion altered the troponin complex's response to Ca²⁺ binding, impaired the sarcomere force generation, and decreased calcium sensitivity.

### Example 2. Overall Structure of ΔK210

To investigate the structural basis for the regulation of Ca²⁺ binding, a three-dimensional structure of ΔK210 bound with Ca²⁺ at 3.1 Å resolution was determined (FIG. 1C and Table 1).

TABLE 1

| | | | | | | | |
|---|---|---|---|---|---|---|---|
| | | | ΔK210- | ΔK210- | ΔK210- | ΔK210- | ΔK210- |
| Ligand | WT | ΔK210 | risedronate | pamidronte | alendronate | ibandronate | neridronate |

Data collection

| | | | | | | | |
|---|---|---|---|---|---|---|---|
| Wavelength (Å) | 0.9795 | 0.9795 | 0.9795 | 0.9795 | 0.9795 | 0.9795 | 0.9795 |
| Resolution range (Å) | 50-3.15 | 50-3.10 | 50-2.60 | 50-3.20 | 50-3.20 | 50-3.20 | 50-3.40 |
| | (3.26-3.13)^{a} | (3.21-3.10) | (2.69-2.60) | (3.31-3.20) | (3.31-3.20) | (3.31-3.20) | (3.52-3.40) |
| Space group | P 2₁ | P 2₁ | P 2₁ | P 2₁ | P 2₁ | P 2₁ | P 2₁ |
| Unit cell (Å, °) | 42.1, | 40.2, | 39.4, | 40.2, | 39.5 | 39.5 | 40.8, |
| | 168.2, | 170.5, | 169.3 | 169.5, | 169.7, | 169.3, | 170.4, |
| | 69.7 | 69.5 | 69.4 | 70.0 | 69.3 | 69.4 | 69.7 |
| | 90, | 90, | 90, | 90, | 90, | 90, | 90, |
| | 101.4, | 101.7, | 102.1, | 101.9, | 102.1, | 102.1, | 101.7, |
| | 90 | 90 | 90 | 90 | 90 | 90 | 90 |
| Total reflections | 129308 | 73788 | 169488 | 66531 | 76955 | 72392 | 58294 |
| Unique reflections | 16901 | 15584 | 26261 | 13714 | 11852 | 13370 | 11980 |
| Multiplicity | 7.7 | 4.7 | 6.5 | 4.9 | 6.5 | 5.4 | 4.9 |
| | (5.9) | (4.4) | (4.7) | (3.7) | (4.8) | (3.9) | (3.1) |
| Completeness (%) | 99.3 | 94.2 | 98.4 | 94.5 | 85.3 | 95.9 | 97.8 |
| | (99.1) | (83.0) | (92.6) | (83.5) | (72.0) | (86.4) | (92.2) |
| Mean I/sigma (I) | 12.6 | 17.7 | 20.3 | 7.8 | 10.1 | 9.1 | 10.0 |
| | (3.0) | (2.7) | (3.1) | (1.6) | (2.6) | (1.4) | (1.5) |
| CC1/2, CC* | (0.483, | (0.903, | (0.992, | (0.921, | (0.974, | (0.970, | (0.967, |
| | 0.807) | 0.974) | 0.998) | 0.979) | 0.993) | 0.992) | 0.992) |
| Rmerge | 0.203 | 0.076 | 0.083 | 0.213 | 0.186 | 0.173 | 0.171 |
| | (1.973) | (0.458) | (0.594) | (0.885) | (0.585) | (0.801) | (1.002) |

Structure refinement

| | | | | | | | |
|---|---|---|---|---|---|---|---|
| R-factor/ R-free^{b} | 0.2296/ | 0.2387/ | 0.2148/ | 0.2599/ | 0.2834/ | 0.2613/ | 0.2553/0.2842 |
| | 0.2638 | 0.2659 | 0.2616 | 0.3264 | 0.3194 | 0.3295 | |
| RMS (bonds) | 0.003 | 0.002 | 0.005 | 0.002 | 0.002 | 0.002 | 0.002 |
| RMS (angles) | 0.647 | 0.486 | 0.922 | 0.552 | 0.448 | 0.532 | 0.450 |
| No. of atoms | 5396 | 5370 | 5427 | 5381 | 5381 | 5381 | 5381 |
| Macromolecules atoms | 5390 | 5364 | 5366 | 5375 | 5375 | 5375 | 5375 |
| Ligands atoms | 6 | 6 | 6 | 6 | 6 | 6 | 6 |
| Water | 0 | 0 | 55 | 0 | 0 | 0 | 0 |
| Average B-factor | 39.1 | 45.0 | 53.9 | 45.9 | 41.9 | 44.5 | 35.8 |

Ramachandran plot statistics

| | | | | | | | |
|---|---|---|---|---|---|---|---|
| Most favored regions | 93.5 | 93.1 | 96.9 | 89.6 | 94.1 | 90.2 | 93.9 |
| (%) | | | | | | | |
| Allowed regions (%) | 5.7 | 6.1 | 3.1 | 9.6 | 5.3 | 8.9 | 5.5 |
| Generously allowed | 0.8 | 0.8 | 0 | 0.8 | 0.6 | 0.9 | 0.6 |
| regions (%) | | | | | | | |
| Disallowed regions (%) | 0 | 0 | 0 | 0 | 0 | 0 | 0 |

^{a}The values for the data in the highest resolution shell are shown in parentheses.
^{b}Rfree = ΣTest||Fobs| − |Fcalc||/ΣTest |Fobs|, where “Test” is a test set of about 5% of the total reflections randomly chosen and set aside prior to refinement for the structure.

Preparations of the troponin core domain similar to those described in Takeda et al., Nature. 2003 Jul. 3; 424 (6944): 35-41, the disclosure of which is incorporated herein in its entirety, were selected for use in the exemplary methods herein; however, different complex assembly strategies and crystallization conditions were applied in the studies of the present disclosure. To eliminate the bias from the purification process and crystallization conditions, a wild-type protein (WT) bound with three Ca²⁺ was also determined at 3.1 Å resolution with the same purification procedure and crystallization conditions as used for the ΔK210 protein and then used as a reference for structure analysis (FIG. 1C and Table 1). The overall structures of both WT and ΔK210 bound with three Ca²⁺ were helical dominated. The ITarm consisted of TnnI and TnnT. TnnC contained two EF handed Ca²⁺ binding domains, of which, the activation Ca²⁺ binding domain bound one Ca²⁺ (designated as “Ca1”) while the structural Ca²⁺ binding domain bound two Ca²⁺ (designated as “Ca2” and “Ca3”). TnnI/T/C engaged in extensive contact with each other to form a stable heterotrimer, where the binding of the switch peptide at the C-terminal of TnnI to the activation Ca²⁺ binding domain of TnnC upon Ca²⁺ association fully activated the whole complex (FIGS. 1C-1D).

The structures of the WT and ΔK210 disclosed herein shared the same dimensions of unit cell with the same space group and the same two protomers of troponin complex per asymmetric unit as the published WT structure (where the “published WT” construct as referred to herein is PDB DOI: 10.2210/pdb1J1D/pdb as disclosed in Takeda et al., Nature. 2003 Jul. 3; 424 (6944): 35-41). The protomer A on the left was more stable than promoter B. Also, the alignment of the two promoters in the asymmetric unit showed that the two protomers in ΔK210 adopted a similar conformation (r.m.s.d 0.853 Å for 304 Cα of 331 Ca) while they were more flexible in the WT structure of the exemplary structures herein and the published WT structures (r.m.s.d 1.295 Å for 271 Cα of 329 Cα in our WT and r.m.s.d 1.552 Å for 288 Cα of 330 Cα in the published WT). The flexibility was due the activation Ca²⁺ binding domain on both protomers which had relative different orientations due to intrinsic flexibility (FIGS. 5B-5D). Thus, the more stable protomer A with the same packing environment for all three structures were selected for further analysis herein. Thermal-shift assays (TSA) were performed to show that ΔK210 was slightly more stable than WT at apo state and at the presence of either Ca²⁺ or Mg²⁺ (Table 2).

TABLE 2

| Protein/Ligand | Ligand free | Ca²⁺ | Mg²⁺ |
|---|---|---|---|
| WT | 42.8 ± 0.5° C. | 48.7 ± 0.5° C. | 46.0 ± 0.1° C. |
| ΔK210 | 43.7 ± 0.3° C. | 50.2 ± 0.2° C. | 47.2 ± 0.5° C. |

### Example 3. In Silico Computational Analysis for WT and ΔK210 Post-Crystallization

Resolved crystal structures of the troponin complex for WT and ΔK210 constructs were analyzed at the levels of contacts between different chains, backbone bond angles, dihedral angles, and calcium binding domains. Generally, slight changes were observed for the interacting hydrophobic, ionic, and hydrogen bond interactions at the levels of contacts between TnnT, TnnI, and TnnC (Table 3) and within the same chain (Table 4).

TABLE 3

| | | |
|---|---|---|
| Interacting | Lost | Newly form in |
| chains | from WT | mutant K^{210del} |

Interacting contact between three chains after refinement

| | | | |
|---|---|---|---|
| Ionic Bond | TnnC with TnnI | GLU126 → | ASP3 → LYS46 |
| | | ARG45 | |
| | TnnT with TnnI | | GLU213 → ARG98 |
| | TnnT with TnnC | | ARG147 → |
| | | | ASP269 |
| Hydrophobic | TnnC with TnnI | PHE27 → | |
| interactions | | MET154 | |
| Hydrogen | TnnT with TnnI | ASN271 → | |
| Bond | | ARG136 | |

Effect of risedronate on the interacting contact
between three chains after refinement

| | | |
|---|---|---|
| Ionic Bond | TnnC with TnnI | ASP3 → LYS46 |
| | TnnT with TnnI | GLU213 → ARG98 |
| | TnnT with TnnC | ARG147 → |
| | | ASP269 |
| Hydrophobic | TnnC with TnnI | |
| interactions | | |
| Hydrogen | TnnT with TnnI | |
| Bond | | |

TABLE 4

| | | |
|---|---|---|
| Interacting | Lost | Newly form in |
| chains | from WT | mutant K^{210del} |

Contact within the same chain after refinement

| | | | |
|---|---|---|---|
| Ionic Bond | TnnI | | LYS21 → GLU10 |
| Hydrophobic | TnnC | PHE20 → MET85 | |
| interactions | | | |
| Hydrogen | TnnC | GLU94 → THR150 | LYS21 → ASP25 |
| Bond | | | SER35 → VAL72 |
| | | | ASP141 → GLY146 |
| | TnnT | | GLU234 → THR238 |
| | TnnI | | ARG111 → GLU115 |

Effect of risedronate on contact within
the same chain after refinement

| | | | |
|---|---|---|---|
| Ionic Bond | TnnI | | |
| Hydrophobic | TnnC | | |
| interactions | | | |
| Hydrogen | TnnC | GLU94 → ARG45 | LYS21 → ASP25 |
| Bond | | | SER35 → VAL72 |
| | | | ASP141 → GLY146 |
| | TnnT | | GLU234 → THR238 |
| | TnnI | | ARG111 → GLU115 |

For the backbone bond angle, TnnT showed a change in the bond angle starting from H222 till Q227, especially at L223 and N224. TnnC showed significant change in the backbone bond angles, especially in the calcium binding amino acids while maintaining the complex to be formed. Bond angle change propagated across TnnI from N-terminal to C-terminal. Regarding the dihedral angle validated by Ramachandran plots, TnnT showed change in the planarity starting from H222 till Q227 (more planar). Afterwards planarity was restored till C-terminal. TnnC showed significant change in dihedral angles, especially in the calcium binding amino acids (D67 and S69). TnnI in the mutant form is less planar compared to WT. However, the crystal structure of Troponin complex for ΔK210+risedronate showed a decrease in the planarity especially from H222 till Q227. Meanwhile, TnnC and TnnI did not show any change from WT.

### Example 4. Allosteric Regulation of Activation Ca²⁺ Binding Domain on TnnC by K210 Deletion

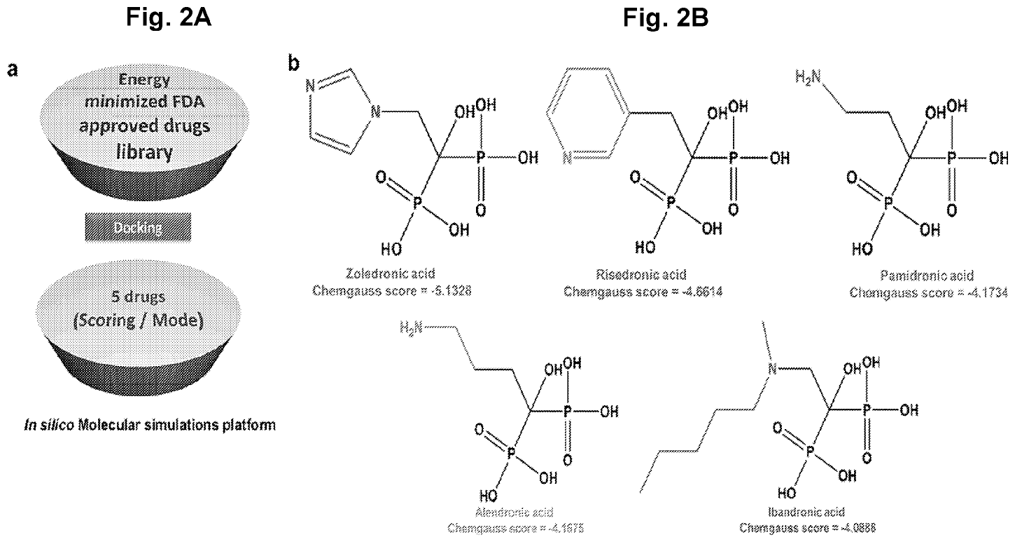

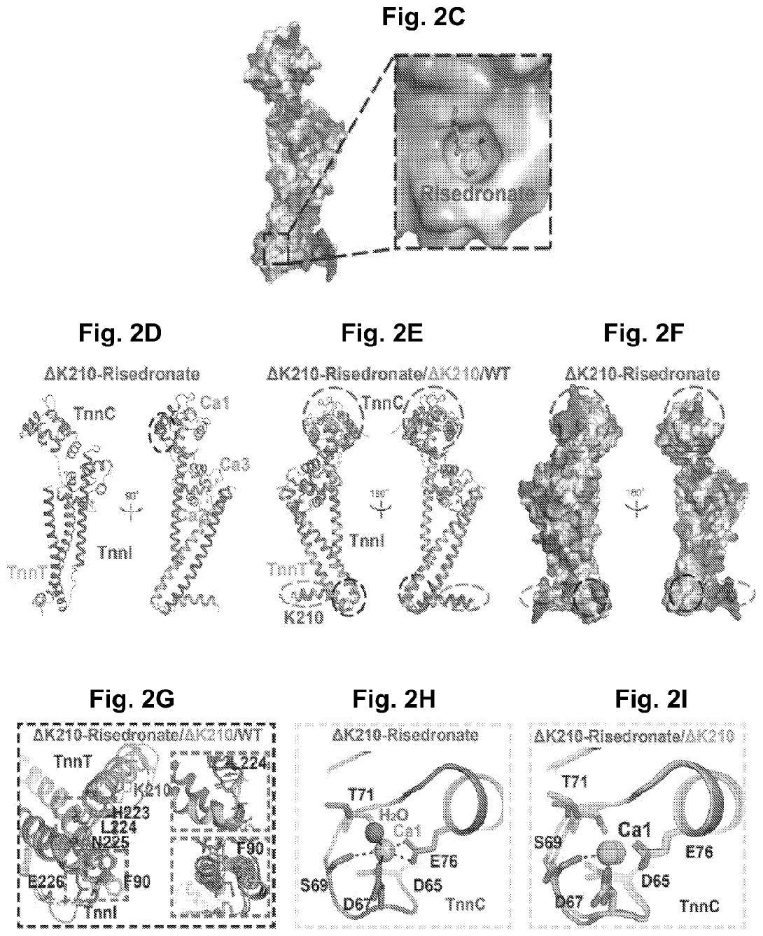

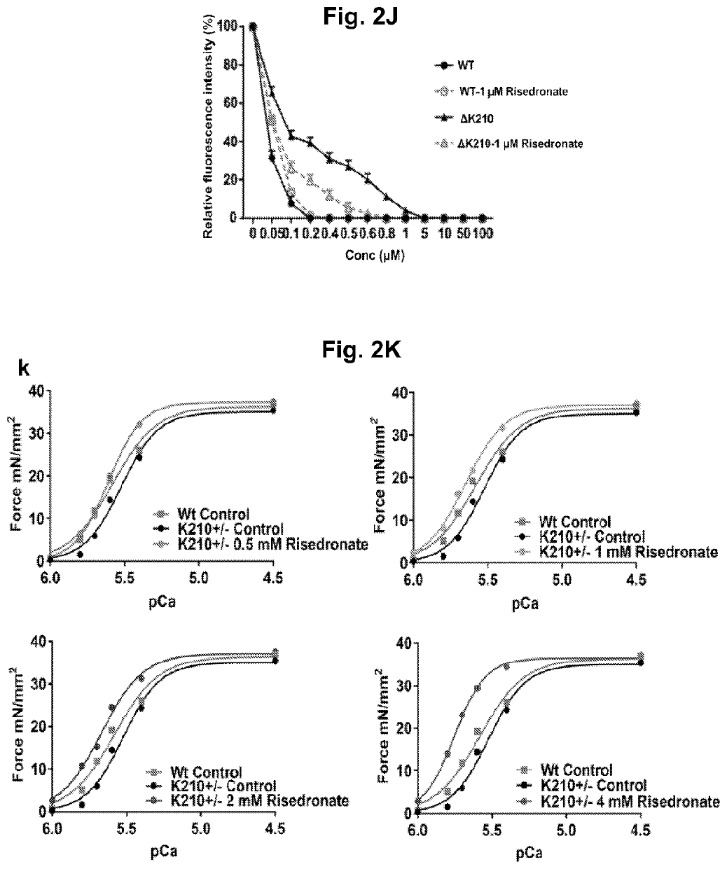

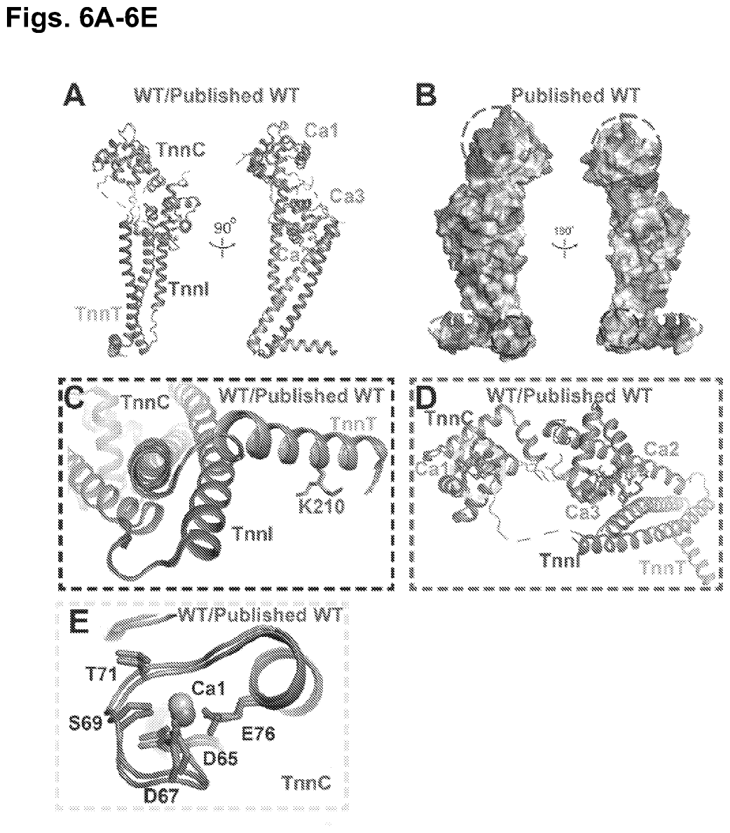

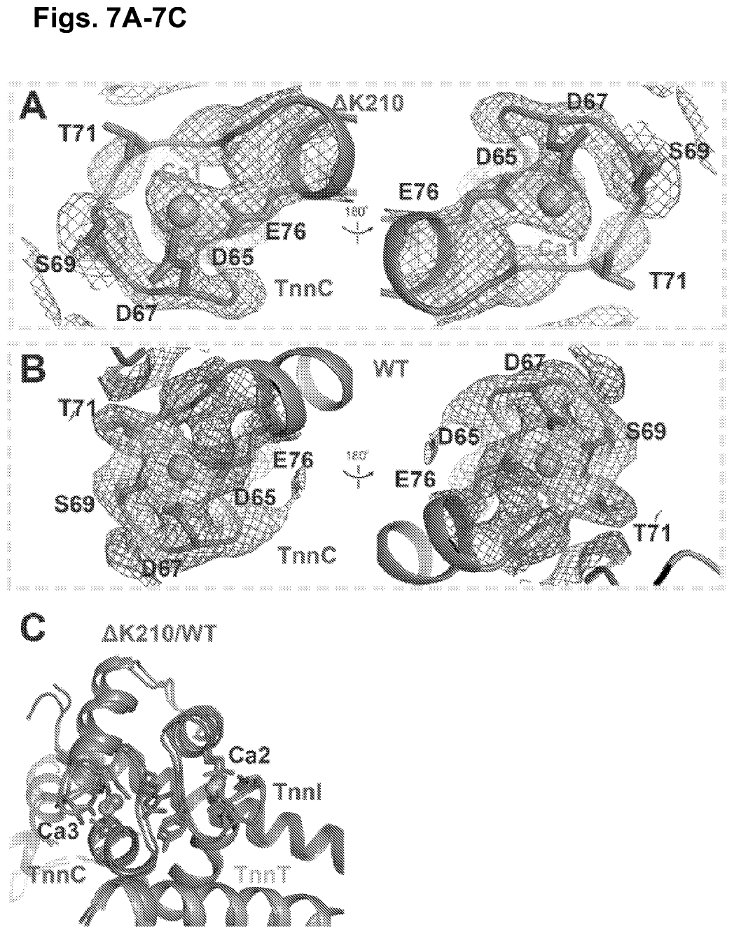

A structure comparison between the exemplary WT structure prepared herein and the published WT (Takeda et al., Nature. 2003 Jul. 3; 424 (6944): 35-41) showed adoption of almost the same conformation (r.m.s.d 0.568 Å for 321 Cα of 339 Ca, FIG. 6A). But compared with either WT construct, the ΔK210 construct had significant differences in three regions: 1) the K210 deletion site; 2) the hinge region (residues from 220-228 in TnnT2 and from 82-91 in TnnI) close to K210 deletion site; and 3) the activation Ca²⁺ binding domain on TnnC away from K210 deletion site (FIG. 2A). Those three regions displayed unique surface topography and electrostatic potential in ΔK210 which was not seen in either WT structure (FIGS. 1E-1F and 6B). Specifically, the surface charge at the site of K210 was greatly altered upon K210 deletion. Additionally, there were two loops moving in the hinge region between WT and ΔK210 while those two loops adopted the exact same conformation in both the exemplary WT structure prepared herein and the published WT (FIGS. 2B-2D and 6B-6C). The movement of these two loops resulted in a newly formed narrow cavity in ΔK210 (See, black dashed circle in FIG. 2C) and slight shift of the whole ITarm (FIG. 2A). Moreover, the activation Ca²⁺ binding domain on TnnC underwent a 4° rotation from WT to ΔK210 due to the alterations in the hinge region and the ITarm while the orientation of the whole domain remained the same in the WT constructs (FIGS. 2E and 6D). Surprisingly, a striking difference was observed between the activation Ca²⁺ binding domain on TnnC of WT compared to that of the ΔK210 construct. In brief, the Ca²⁺ coordination network of ΔK210 was distorted upon K210 deletion and the S69 on TnnC of ΔK210-which was involved in the coordination with Ca²⁺ in WT—was flipped away, leading to the loss of one bond for Ca²⁺ coordination (FIGS. 1I-1K, 6E, 7A, and 7B). The structural Ca²⁺ binding domain had no obvious change between WT and ΔK210 (FIG. 7C). Moreover, the average length of bonds for the other coordinated residues with Ca²⁺ in ΔK210 (average of 2.9 Å for 5 hydrogen bonds) were longer than the bonds in WT (average of 2.6 Å for 6 hydrogen bonds) (FIGS. 1I-1J). Also, Ca²⁺ binding free energy analyzed by MOE suggested that the binding free energy of Ca²⁺ to the activation Ca²⁺ binding domain in ΔK210 (−1.77 kcal/mol) was higher than in WT (−2.80 kcal/mol). The analysis also showed that the binding free energy of Ca²⁺ to the structural Ca²⁺ binding domain in both structures was very similar (−3.50 kcal/mol) which was lower than that of activation Ca²⁺ binding domain and agreed with the higher binding affinity. Therefore, K210 deletion on TnnT induced local change on TnnT and TnnI and allosterically regulated the conformation of the activation Ca²⁺ binding domain and the hydrogen bond network of Ca²⁺ coordination on TnnC.

### Example 5. In Silico and In Vitro Pharmacological Correction of ΔK210 Defect

A structure-based drug repurposing approach to identify FDA approved drugs targeting ΔK210 was used similar to that described in Ahmed et al., Proc Natl Acad Sci USA. 2021 Mar. 9; 118 (10): e2016265118, the disclosure of which is incorporated herein in its entity. In brief, the MMFF94 energy-minimized library of FDA approved drugs were prepared and docked to the hinge region in ΔK210 (FIG. 2A). The screening identified five drugs belonging to the bisphosphonates therapeutic class based on their binding energies and interacting profiles (FIG. 2B). The drugs of the bisphosphonates family were found out to bind towards the induced hinge motif at (HLNEDQLR; SEQ ID NO: 1) via hydrophobic interactions and hydrogen bond interactions. Of the five drugs, risedronate acid showed proper binding mode regarding energy scoring towards the hinge region of ΔK210 (FIG. 2C).

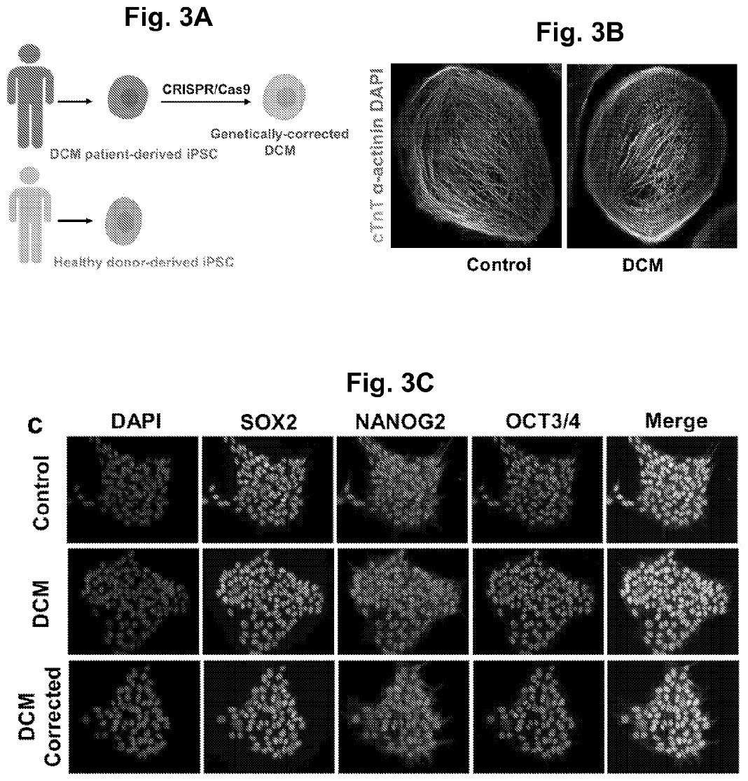

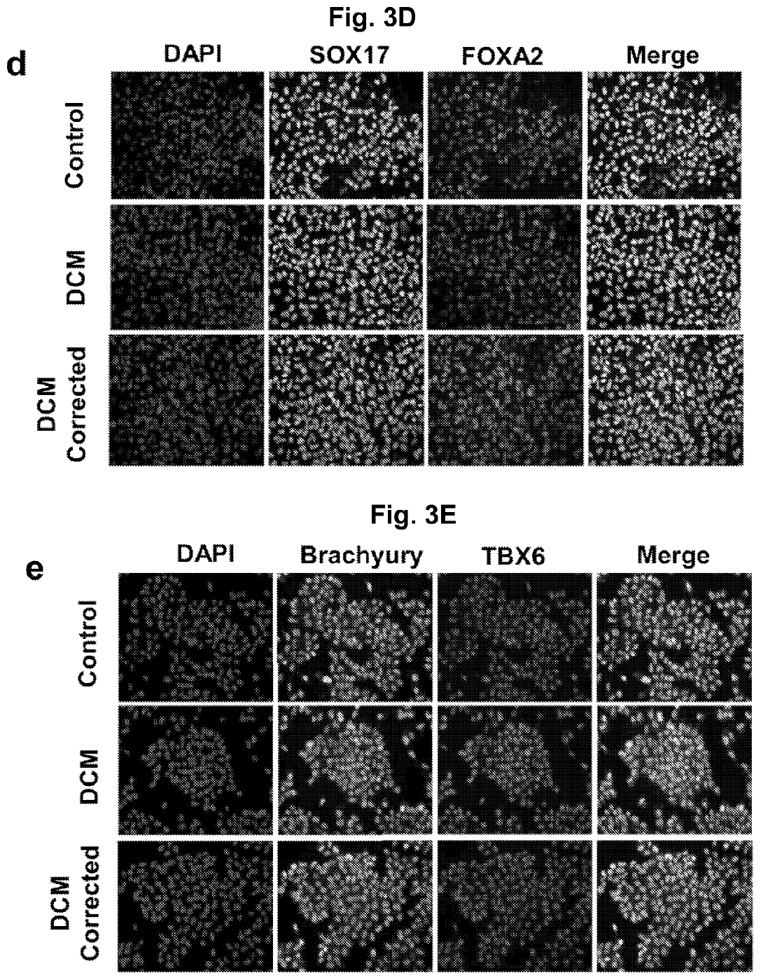

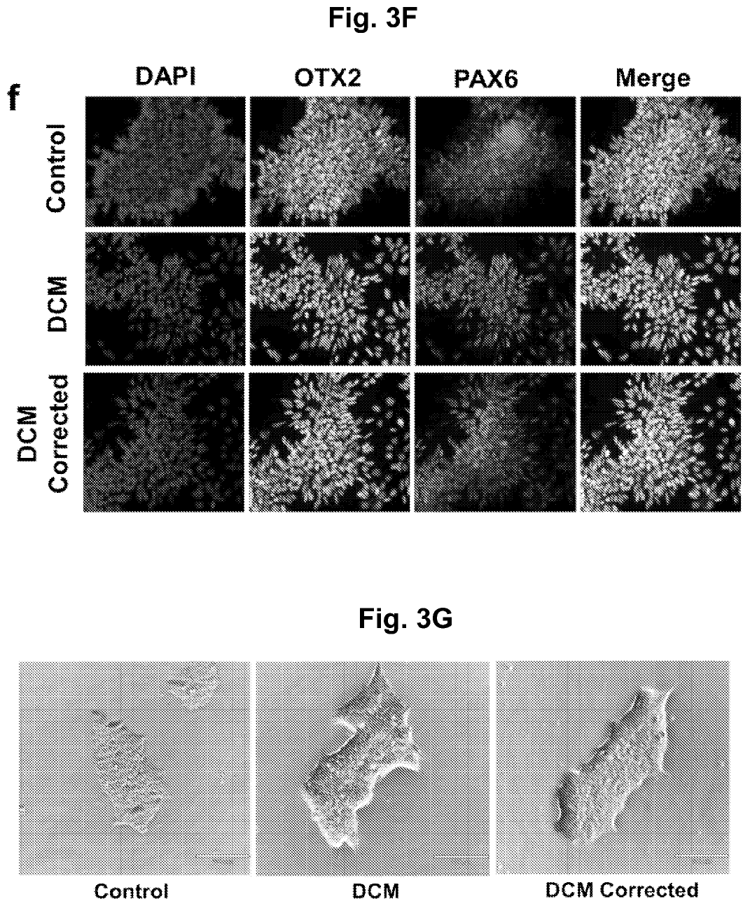

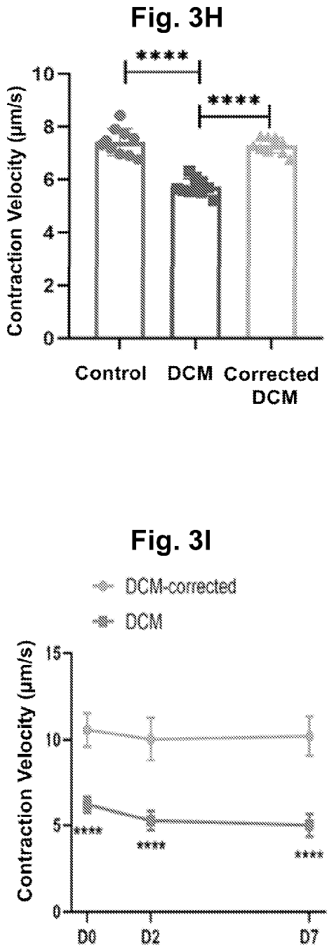

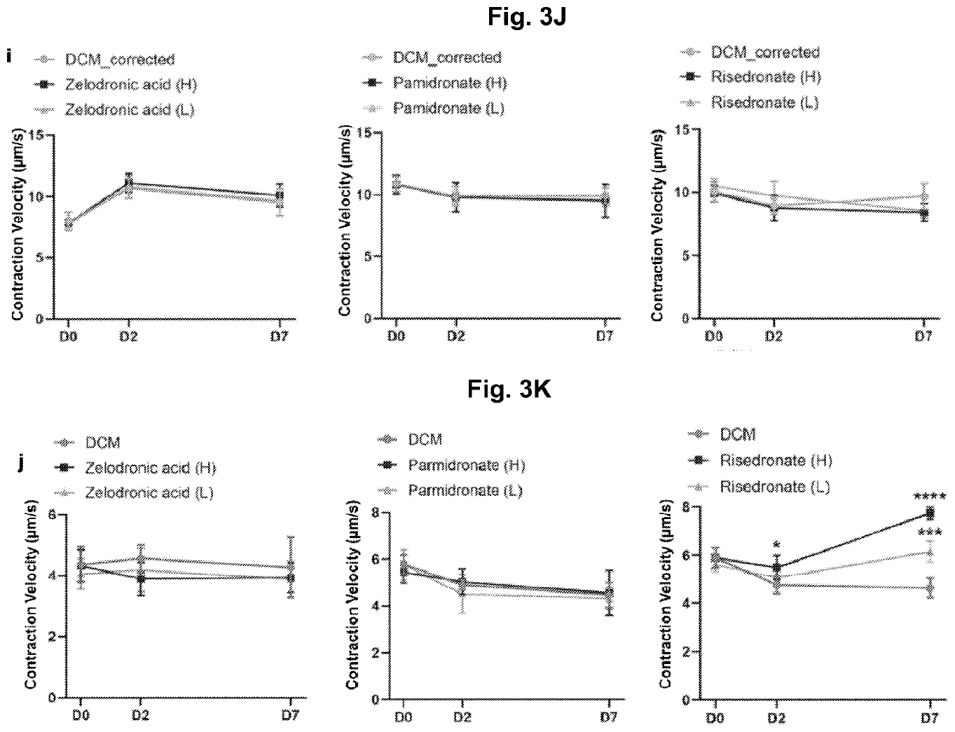

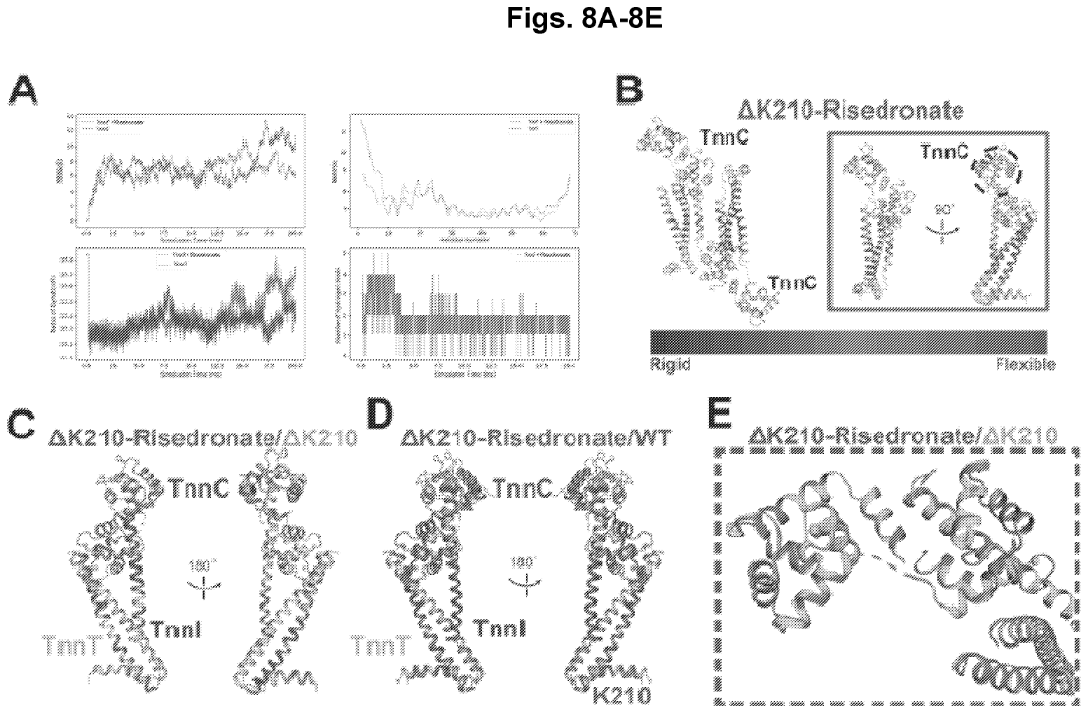

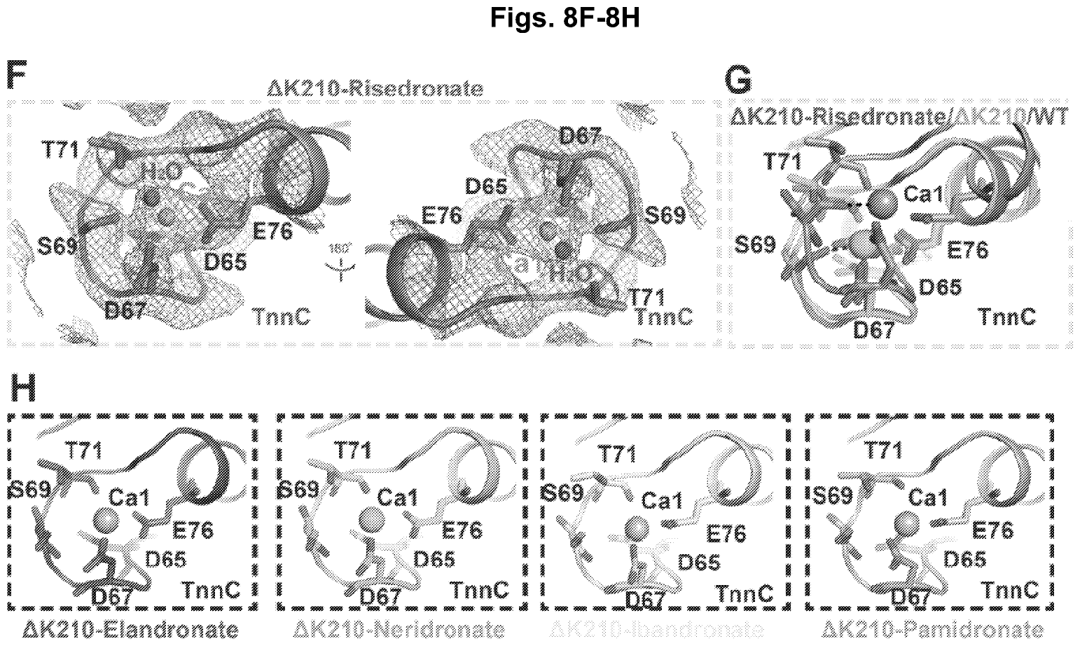

ΔK210 bound with Ca²⁺ in the presence of risedronate (ΔK210-risedronate hereafter) was resolved at 2.6 Å to elucidate how risedronate might alter the structure. The more stable protomer A was selected for further analysis (FIGS. 8A-8B). The overall structure of ΔK210-risedronate also resembled an active form of TnnT complex and was more closed to ΔK210 in the absence of risedronate than WT (FIGS. 2D-2F, 8C, and 8D). Significant differences were observed in the structure of ΔK210-risedronate compared with ΔK210 in the absence of risedronate. First, the surface topography and electrostatic potential were slightly different compared to both WT and the ΔK210 in the absence of risedronate, especially in the hinge region (FIG. 2F). The two loops consisted of the hinge region in the structure of ΔK210-risedronate moving toward the conformation of WT, although not exactly the same as that observed for WT. The side chain of L223 in TnnT and F90 in TnnI were flipping to the same conformation as WT (FIG. 3G). The overall orientation of both Ca²⁺ binding domain had no significant change compared with ΔK210 by itself (FIG. 8E), but the activation Ca²⁺ binding pocket had some local conformational change due to the allosteric effect on the hinge region. Specially, the loop containing S69 had a mild shift toward Ca²⁺ and the side chain of S69 was flipping inward to reestablish the coordination with Ca²⁺ (FIGS. 2H-2I, 8F, and 8G). The Ca²⁺ coordination network in the structure of ΔK210-risedronate also had preferable Ca²⁺ binding free energy (−XXX kcal/mol). Meanwhile, the structure of ΔK210 in the presence of the other family members of bisphosphonate such as elandronate, ibandronate, neridronate and pamidronate were resolved in the same crystallization condition as ΔK210 in the presence of risedronate (Table 1). No change was observed in the hinge region and activation Ca binding domain in either of these structures compared with ΔK210 alone (FIG. 8H) while no ligand density was observed. Collectively, the presence of risedronate could alter the Ca²⁺ binding affinity through affecting the conformation of hinge region and allosterically correct the defect of the activation Ca²⁺ binding network caused by K210 deletion. A force generation assay using skinned papillary muscle and a fluorescence reporter assay were performed to show the ability of risedronate to increase the maximal force produced at low calcium concentrations. Results showed that the improved the Ca²⁺ binding induced fluorescence change (FIGS. 2J-2K).

### Example 6. Ex-Vivo Pharmacological Evaluation for K210Del Mediated Dilated Cardiomyopathy Using iPSC-CM

Next, healthy, DCM patient-specific iPSCs showing K210del, and genetically corrected patient specific iPSCs were collected and reprogramed from human peripheral blood mononuclear cells (PBMCs) to account for their morphology, pluripotency, trilineage differentiation across endoderm, mesoderm, and ectoderm (FIGS. 3A-3G). Then, the contraction velocity was tested showing the ability of genetically corrected DCM to restore contraction, compared to DCM patient-derived iPSCs (FIGS. 3H-3I). Next, the potential of bisphosphonate family members (zoledronate, pamidronate, and risedronate) to restore the contraction velocity for DCM patient-derived iPSCs and genetically corrected DCM was tested. Risedronate showed a significant increase in the contraction velocity for DSM patient-derived iPSCs (FIG. 3J). However, the three drugs did not show any change in the contraction velocity at genetically corrected DCM (FIG. 3K). This suggested that risedronate could be plausible therapeutic to enhance contraction velocity in K210 del mediated DCM.

### Example 7. In Vivo Pharmacological Evaluation of Risedronate K210Del DCM Mouse Model

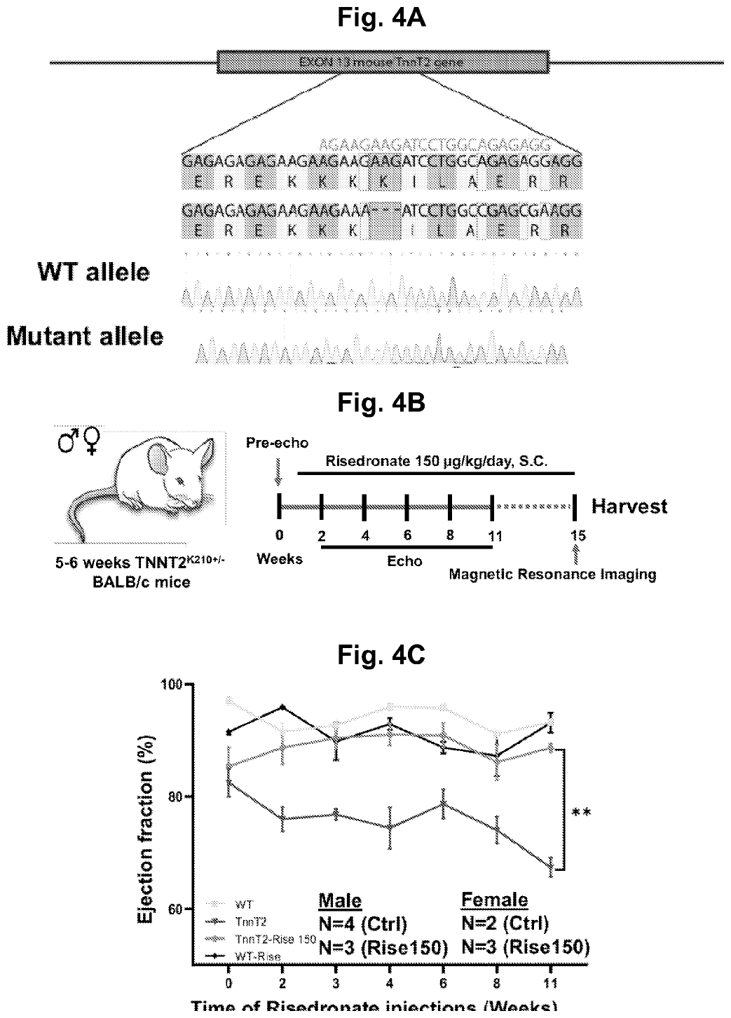

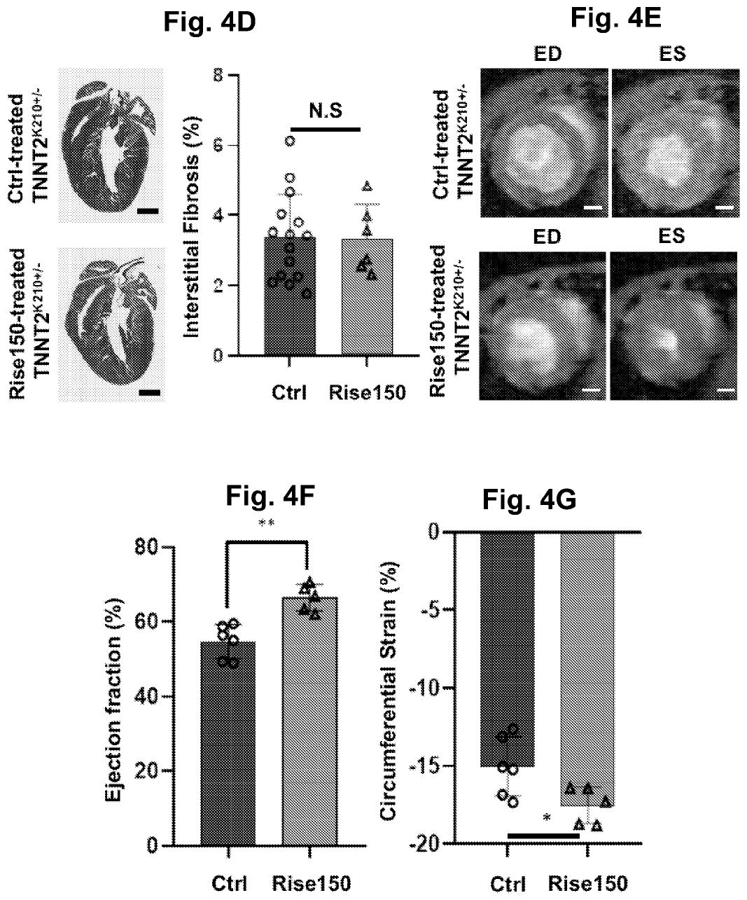

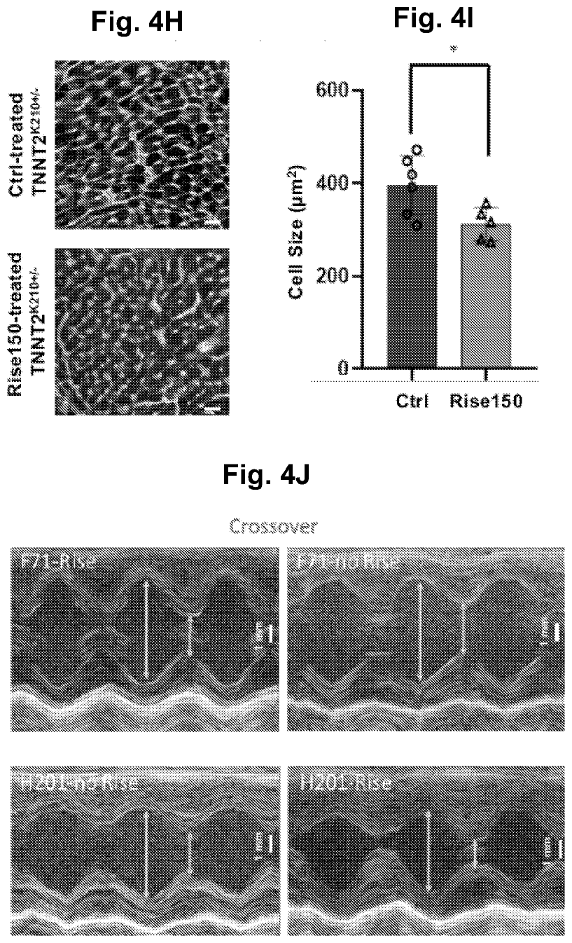

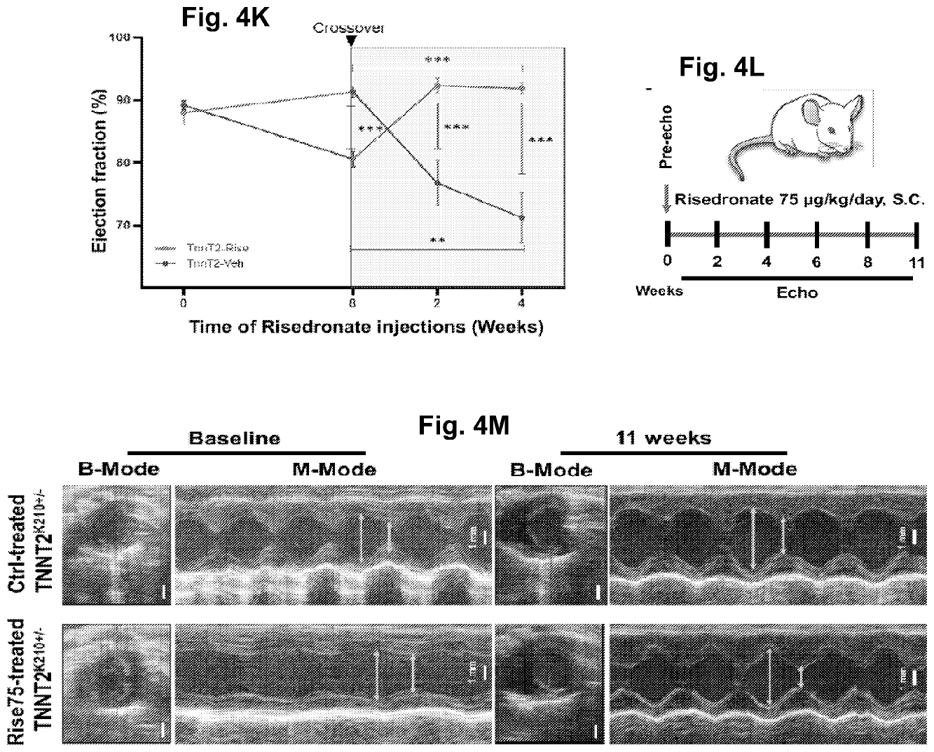

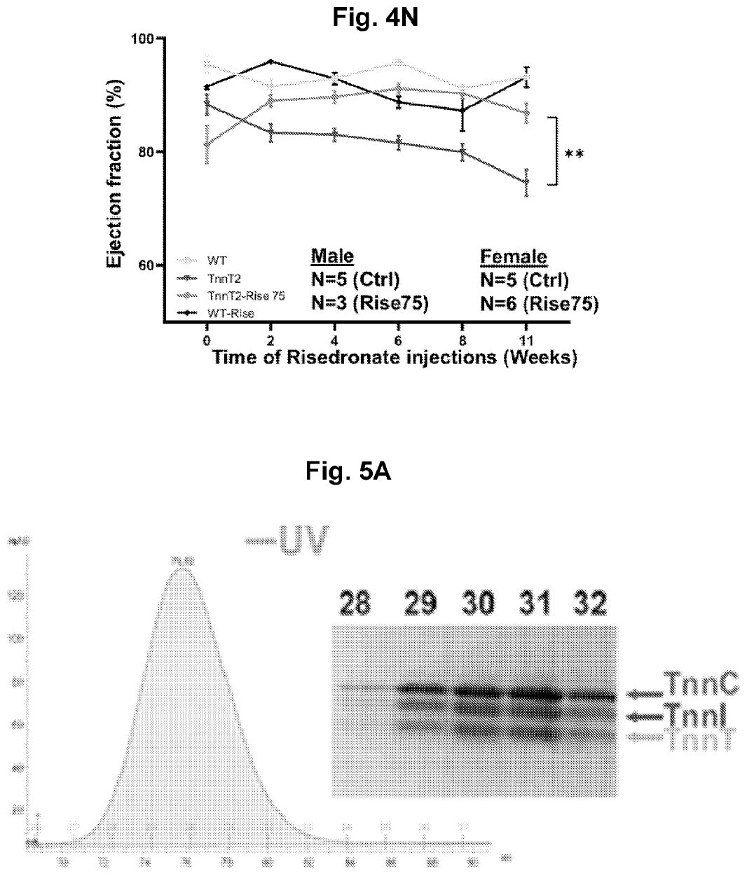

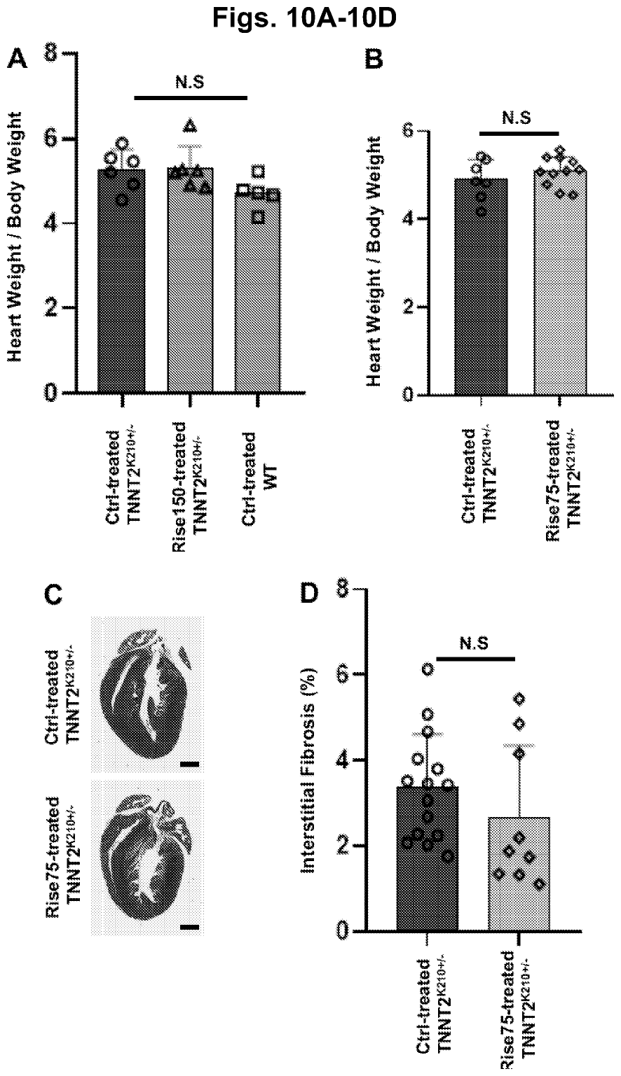

To test the therapeutic potential of risedronate to correct the DCM associated with the deletion mutation ΔK210 in cTnnT, a well-established mouse model was used (FIG. 4A). In this exemplary study, echocardiography was conducted for 5-6 weeks in TnnT^{K210}+/−BALAB/C mice prior to the administration of Risedronate (150 μg/kg/day, S.C.) for 15 weeks while accounting for left ventricular ejection fraction (LVEF %) using echocardiography and magnetic resonance imaging (MRI) (FIG. 4B). Heart to body weight for TNNT2^{K210+/-}, risedronate-treated TNNT2^{K210+/-} (150 μg/kg/day), and TNNT2WT were determined and provided in FIG. 10A. Risedronate showed a significant increase in the ejection fraction over the entire-course of administration compared to control-treated K210+/− mice (FIG. 4C). This was further confirmed by magnetic resonance imaging (MRI) which showed a significant increase in the LVEF and lower circumferential strain with a similar heart rate in the risedronate-treated TNNT2^{K201+/-} mice compared to control-treated TNNT2^{K201+/-} mice (FIGS. 4D-4G). Risedronate-treated TNNT2^{K201+/-} mice did not show significant changes in the interstitial fibrosis (FIG. 4D), however they showed significant reduction in the cell size after WGA staining, compared to control-treated TNNT2^{K201+/-} mice (FIGS. 4H-4I). This was followed by conducting a cross-over study to test the ability of risedronate to reverse the decline of LVEF % after 8-weeks of risedronate and control treatment after surrendering decline in the LVEF % for the control-treated TNNT2^{K210+/-} mice and extending the study for 4-weeks. This showed that risedronate restored the increase in LVEF % significantly after 4 weeks of administration (FIGS. 4J-4K).

Another dose level for risedronate was tested to mimic the administered therapeutic dose in human, where Risedronate (75 μg/kg/day, S.C.) was administered for 5-6 weeks to TnnT^{K210+/-}BALAB/C mice. Heart to body weight for TNNT2^{K210+/-}, risedronate-treated TNNT2^{K210+/-} (175 μg/kg/day), and TNNT2WT were determined and provided in FIG. 10C. Masson's trichrome staining of hearts, 6-weeks post-risedronate administration (75 μg/kg/day) in TNNT2^{K210+/-} mice, showing non-significant change in the interstitial fibrosis, compared with control-treated TNNT2^{K210+/-} hearts (FIGS. 10C-10D). Risedronate-treated TnnT2^{K210+/-} mice still had increased LVEF % compared to control-treated TNNT2^{K201+/-} mice (FIGS. 4L-4N). These results suggested that risedronate improves LV systolic function for DCM resulted from K210del in TNNT.

### Methods Used in Examples 1-7

Provided herein are exemplary methods and exemplary materials used in the examples of the present disclosure detailed as follows:

Protein expression and purification. hTnnT2 with residues 183-288 and hTnnI with residue 32-166 were subcloned into the first and second Open Reading Frame (ORF) of pRSFDuet vector, respectively and hTnnC with residues 1-161 was subcloned into the first ORF of pETDuet vector with N terminal 6×His tag followed by TEV protease recognition site. Both plasmids were transformed into Rosetta (DE3) pLysS cells (Novagen). According to the previous structure disclosed in Takeda et al., Nature. 2003 Jul. 3; 424 (6944): 35-41, the cysteine-less variant TnnC (C35S/C84S) and the variant TnnI (T31M/C80A/C97A) were used in the exemplary methods herein. For the K210 deletion mutation (ΔK210), the cDNA was synthesized by Genescript and further subcloned into the first ORF of pRSFDuet vector for later co-transformation. Target protein was expressed in cultures grown in autoinduction media at 18° C. overnight. The culture was harvested and sonicated in lysis buffer (50 mM Tris (pH8.0), 150 mM NaCl, 1 mM DTT, 1 mM CaCl₂) and supplemented with protease inhibitors). The lysate was centrifuged, the supernatant was loaded onto a Ni-NTA affinity column (Qiagen) and the beads were washed with wash buffer (20 mM Tris (pH8.0), 150 mM NaCl, 1 mM DTT and 20 mM Imidazole (pH 8.0)) and eluted with elution buffer (20 mM Tris (pH 8.0), 150 mM NaCl, 1 mM DTT and 250 mM imidazole (pH 8.0)). The eluate was digested by TEV protease at 4° C. overnight and then further purified by ion exchange chromatography followed by gel filtration chromatography. The peak fractions were collected and concentrated to about 25 mg/mL for crystallization screening with the gel filtration buffer (20 mM Tris (pH 8.0), 150 mM NaCl, 1 mM DTT and 1 mM CaCl₂)).

Crystallization and structure determination. The crystals of the TnnT2/TnnI/TnnC wild type and ΔK210 mutant were obtained using the hanging-drop, vapor-diffusion method by mixing 1 μL protein (25 mg/mL) with 1 μL reservoir solution containing 0.2 M sodium acetate and 20% PEG 3350 and incubating at 18° C. The crystals were observed after three days and reached the maximum size after seven days. The crystals were flash frozen in liquid nitrogen with 15% glycerol as the cryo protectant. The datasets were collected at APS-19-ID at wavelengths of 0.97926 Å. Data was indexed, integrated and scaled by the program HKL3000. Phases were determined by molecular replacement using the wild type TnnT2/TnnI/TnnC structure (PDB code: 1J1D) as a searching model. The model was further built manually with COOT (Emsley & Cowtan, Acta Crystallogr D Biol Crystallogr 60, 2126-2132 (2004)) and iteratively refined using Phenix.refine (Adams et al., Acta crystallographica. Section D, Biological crystallography 66, 213-221 (2010)). The PROCHECK program was used to check the quality of the final model, which shows good stereochemistry according to the Ramachandran plot (Laskowski et al., Journal of Applied Crystallography 26, 283-291 (1993)). All structure figures were generated by using the PyMOL Molecular Graphics System, Schrödinger, LLC. Software used in this project was curated by SBGrid (Morin et al. Elife 2, e01456-e01456 (2013)).

In silico molecular docking simulations. The U.S. Food and Drug Administration approved drug database was downloaded and three-dimensional (3D) structures were energy minimized using MMFF94 force field. The whole library underwent multi-conformer generation. The crystallized ΔK210 mutant model was used as a template structure via placing a box around the induced conformational changed site (LNEDQLR; SEQ ID NO: 2) to be docked with energy minimized FDA library via using Molecular Operating environment (MOE). The scoring assessment was conducted by validating the docked poses and estimating the energy scores. Three-dimensional visualization was conducted using pymol.

In silico computational analysis for WT and ΔK210 post-crystallization. Crystal structures of Troponin complex for WT and K210del were analyzed at the levels of contacts between different chains, backbone bond angles, dihedral angles, and calcium binding domain using Molecular Operating environment (MOE). Z scores (cut-off values) were adjusted to satisfy the minor changes all over the whole complex.

Thermal Shift Assay. The samples for TSA were treated with both 3 mM EGTA and 3 mM EDTA during ion exchange purification and stored in the gel filtration buffer without CaCl₂). Solutions containing 5 μL of 1 mg/mL protein, 9 μL of TSA buffer [20 mM Tris (pH 8.0), 150 mM NaCl, 1 mM DTT and 1 mM CaCl₂)] and 1 μL of SYPRO Orange (diluted 1/25 in water, purchased from Sigma) were added to PCR tubes and the final volume for the reaction was 15 μL. The PCR tubes were heated in a i-Cycler iQ5 real time PCR detection system (Bio-Rad) from 30° C. to 95° C. with an increment of 1° C. The fluorescence signal for each tube were monitored by a charge-couple (CCD) camera. The melting temperature for the protein unfolding transition was analysis by the build-in melt curve calculation mode.

Determination of Ca²⁺ binding induced fluorescence change. The steady-state fluorescence measurements were carried out as previously described in the context of WT^{F27W} and ΔK210^{F27W}. Briefly, the fluorescence emission of WT^{F27W} and ΔK210^{F27W} were measured during Ca²⁺ titration by using an excitation wavelength of 276 nm and an emission wavelength of 340 nm. The fluorescence change upon Ca²⁺ binding was determined by subtracting the fluorescence at maximal Ca²⁺ concentration from all other measurements and then expressing the resultant values as percentages of the maximum fluorescence. The data was plotted by GraphPad.

Reprogramming of iPSCs. The study was carried out under Stanford Institutional Review Board and Stem Cell Research Oversight Committee guidelines. Healthy and Patient-specific iPSCs were collected and reprogramed from human peripheral blood mononuclear cells (PBMCs) using CytoTune-iPS 2.0 Sendai Reprogramming Kit (Thermo Fisher Scientific) and cultured on feeder-free Matrigel-coated culture plates.

Cell culture. Human iPSCs were routinely grown on Matrigel-coated (Corning) 6-well plates using chemically defined E8 medium (Thermo Fischer Scientific) and passaged at a ratio 1:12 every 4 days using Accutase solution (Sigma-Aldrich). hiPSC-CMs were maintained in a RPMI 1640 medium (Thermo Fisher Scientific) supplemented with B27 supplements (Thermo Fisher Scientific). The cells were maintained in the incubator with 5% CO₂ at 37° C.

Trilineage differentiation. The pluripotency of the hiPSCs were assessed by differentiating into three germ layers. The Human Pluripotent Stem Cell Functional Identification Kit (R&D Systems) was used to differentiate hiPSCs into mesoderm and ectoderm, and the STEMdiff Definitive Endoderm Kit (STEMCELL Technologies) was used to differentiate hiPSCs into endoderm.

Immunofluorescent staining. Cells were fixed in 4% paraformaldehyde (PFA) solution (Thermo Fisher Scientific) for 10 minutes and permeabilized with 0.1% Triton X-100 (Sigma Aldrich) in PBS (Thermo Fisher Scientific) for 10 minutes at room temperature (˜ 23° C.+3° C.). Cells were then blocked with 3% bovine serum albumin (BSA) (Sigma Aldrich) in PBS for 30 minutes, followed by overnight incubation at 4° C. with 1% BSA solution containing a 1:200 dilution of primary antibodies. Cells were washed three times with 0.1% Tween-20 (Sigma Aldrich) in PBS and then followed by an incubation with 1% BSA solution containing secondary antibodies for 60 minutes at room temperature in the dark. Nuclei were counterstained with NucBlue Fixed Cell ReadyProbes Reagent (Thermo Fisher Scientific).

Karyotyping. hiPSCs chromosomal aberrations were detected using the KaryoStat assay (Thermo Fisher Scientific).

Sequencing. DNA was extracted from hiPSCs using DNeasy Blood & Tissue Kits (Qiagen). PrimeSTAR® GXL DNA Polymerase (Clontech) was used for PCR. The sequence of primers used for genotyping is as follows: forward primer, 5′-TGATCCTTCTTGCCCCTACCT (SEQ ID NO: 3); and reverse primer, 5′-TTCTTGCTGTGAGCCACCAGA (SEQ ID NO: 4).

Differentiation of hiPSCs into hiPSC-CMs. hiPSCs were split at 1:12 ratio using Accutase solution (Sigma-Aldrich). When hiPSCs reached a confluency of 80%, the E8 medium was changed to RPMI supplemented with B27 without insulin (Life Technologies) and 6 μM of the glycogen synthase kinase 3-β inhibitor CHIR-99021 (Selleck Chemicals) for 2 days (D0-D1). On day 2, the medium was aspirated and replaced with RPMI+B27 minus. On day 3, the medium was changed to RPMI+B27 minus with 5 μM of the Wnt inhibitor IWR-1 (Selleck Chemicals) for 2 days. The medium was replaced with RPMI−B27 minus for 2 days on day 5, and then switched to RPMI−B27 for 3 days on day 7. On day 10, the medium was changed to RPMI−B27 without D-glucose (Life Technologies) for 3 days. This glucose starvation step further purified cardiomyocyte culture. After 2 days' recovery using the medium RPMI−B27, cells were dissociated after a 5-minute incubation with TrypLE Select Enzyme (10×) (Thermo Fisher Scientific) at 37° C. followed by seeding into 6-well plates cultured with RPMI−B27 with 10% Knock-Out Serum Replacement (KOSR) (Thermo Fisher Scientific). 1 day later, the medium was changed back to RPMI−B27. hiPSC-CMs were cultured in RPMI−B27 for experiments after the second purification as described above.

Drug treatment. Human iPSC-CMs were treated with FDA approved candidate drugs for 2 and 7 days. DMSO (Sigma-Aldrich) was used as a control treatment unless noted. Cells were treated with Zoledronic acid at 30 or 300 ng/ml, or Pamidronate at 100 or 1000 ng/ml, or Baclofen at 100 or 1000 ng/ml, or Fenoprofen at 10 or 50 μg/ml, or Risedronate at 2 or 10 ng/ml. The medium containing fresh drug was replaced every 2 days.

Contractility analysis. hiPSC-CMs were dissociated using TrypLE Select Enzyme (10×) (Thermo Fisher Scientific) for 5 minutes at 37° C. Afterwards, cells were seeded on Matrigel-coated 96-well plates (50,000 cells per well) and cultured for 7 days to recover their synchronous beating before the assay. Phase-contrast cell motion movies of cardiomyocyte contraction were recorded using the Sony SI8000 cell motion imaging system (37° C. and 5% CO₂). Prior to data collection, the cells were equilibrated for 15 minutes. Video images of the hiPSC-CMs were recorded for 10 seconds, at a frame rate of 75 frames per second (fps), and a resolution of 1,024×1,024 pixels using a 10× objective. Data analysis was performed using SI8000C Analyzer software (Sony).

Preparation of skinned papillary muscle fiber. 12-14 weeks old mice were euthanized in accordance with approved protocols and complied with the relevant ethical regulations regarding animal research. Hearts were isolated and placed in the chilled PBS buffer. Hearts were subjected to dissection along the aorta line to divide it in left and right ventricles. A pair of papillary muscles were isolated and placed in the skinning buffer, to skin overnight at 4° C. Skinning buffer comprised of 1% Triton X-100 in buffer containing; 55.74 mM potassium propionate, 7 mM ethylene glycol bis(2-aminoethyl) tetra acetic acid, 100 mM N,N-bis(2-hydroxyethyl)-2-amino ethane sulfonic acid, 20 μM calcium chloride, 5.5 mM magnesium chloride, 5 mM dithiothreitol, 15 mM creatine phosphate and 4.7 mM adenosine triphosphate with pH adjusted to 7.0 with 4 M potassium hydroxide and ionic strength maintained at 180 with potassium propionate. The calcium strength in the above buffer was minimal with pCa 9.0 also termed as relaxing buffer since the muscles do not contract in the pCa 9.0 buffer. After overnight skinning, the fibers were ready for experimental use.

Calcium dependent force generation in skinned papillary muscles. The skinned papillary muscle fibers were dissected in fibers of width ˜100 μM and then mounted between a high-speed length controller (Aurora Scientific 322C) and force transducer (Aurora Scientific 403A) using aluminum T-clips. The fibers were cycled through buffers containing increasing amounts of calcium concentration; pCa 6.0, 5.8, 5.7, 5.6, 5.4 and 4.5. All the pCa solutions were made by mixing the relaxing buffer (pCa 9.0) and activating buffer (pCa 4.5). The activating buffer (pCa 4.5) was the same as relaxing buffer except for 7 mM calcium chloride. The fibers cycling through pCa solutions were allowed to achieve saturating maximal force before subjected to the next pCa buffer. Risedronate was added to all the pCa solutions to assess effect on calcium dependent force generation in wild type vs the TNNT2 K210 mutant mice skinned cardiac papillary fibers. 100× stock of risedronate was diluted to its experimental concentration to avoid excessive dilution of the pCa solution. For control experiments, PBS was added in the same amount risedronate in pCa solution to offset any effects due to dilution. The comparison of calcium dependent force generation and calcium sensitivity across control and test experiments was calculated by plotting maximal force generated against the respective pCa. The data was fitted to sigmoidal dose response curve with variable slope (GraphPad Prism 9).

Animals. All mouse experiments were conducted in accordance with approved protocols and complied with the relevant ethical regulations regarding animal research. Mice were housed in a 12:12 hour light:dark cycle in a temperature-controlled room with free access to water and food. Littermate controls were used whenever possible. K210^{+/-} BALB/C mice were used in the whole study. No statistical methods were used to predetermine sample size. All echocardiographic studies were carried out blinded to the treatment of the mice during the experiments and outcome assessments.

Drug dose(s) and administration. Risedronate doses were calculated based on allometric scaling among species. The therapeutic regimen used in the exemplary methods herein was designed to mimic the administered human dose ranges for Risedronate. HED (mg/kg)=Animal dose (mg/kg)*(Animal Kₘ/Human Kₘ)

Transthoracic echocardiography. Assessment of in vivo heart function on conscious, non-sedated mice was performed using a Vevo2100 micro-ultrasound system, MS400C probe (VisualSonics) at baseline, 2 weeks after drug administration, and 4-, 6-, 8-, and 11-weeks drug administration. Echocardiographic M-mode images were obtained from a parasternal short-axis view at the level of the papillary muscles. Left ventricular internal diameters at end-diastole (LVIDd) and end-systole (LVIDs) were measured from M-mode recordings. Six representative contraction cycles were selected for analysis, and average indexes (LVIDd, LVIDs, and fractional shortening) were calculated for each mouse. All echocardiography measurements were performed in a blinded manner.

Cardiac magnetic resonance imaging (MRI). MR imaging was performed on a 7T pre-clinical scanner (Bruker Biospec, Germany) using a 72 mm volume transmitter coil with a 2×2 phased array surface receiver coil. Mice were anesthetized with 1.5-2.5% Isoflurane. The animal's ambient temperature was maintained at 28° C. using an MR Compatible Small Rodent Air Heater System (SA Instruments, Stony Brook, NY). Imaging was performed with prospective gating for ECG (SA Instruments, Stony Brook, NY) monitored using needle electrodes connected to front paws. Cine images in the short axis plane were obtained using a gradient echo (FLASH) sequence. The following imaging parameters were used: TE/TR=3.9/10 ms; number of k-space lines per R−R=1; slice thickness=1 mm, number of averages=3; flip=15°; FOV=30×30 mm²; matrix=192×192; in-plane resolution=0.15×0.15 mm³. Five to 6 contiguous slices were obtained.

MR tagging was performed on a short axis slice at the mid ventricular level using the SPAMM method. Imaging parameters were: TE/TR=4/15 ms; tag separation=1 mm; tag thickness=0.2 mm; number of k-space lines per R−R=1; slice thickness=1 mm, number of averages=3; flip=20°; FOV=30×30 mm²; matrix=192×192; in-plane resolution=0.15×0.15 mm³. Cardiac cine images were analyzed using the freely available software, Segment version 3.0. Myocardial strain analysis was performed using HARP (Diagnosoft Plus, Diagnosoft Inc., CA, USA). Ventricular end diastolic volumes, LV ejection fraction, mean myocardial Eularian circumferential strain (Ecc) were calculated.

Histology. Mouse hearts were collected and fixed in 4% paraformaldehyde in PBS overnight at 4° C. and then processed for paraffin embedding. H&E and Masson's trichrome staining were performed according to standard procedures on paraffin sections.

WGA staining and cardiomyocyte size quantification. WGA staining and quantification were performed as previously described. In brief, the slides were incubated with WGA conjugated to Alexa Fluor 488 (50 mg ml⁻¹, Life Technologies) for 1 hour at room temperature following washing with 1×PBS. To quantify the cross-sectional cell size, three to five independent hearts per group with three different views and positions, each from left and right ventricles, and septum were captured at 40× magnification. ImageJ was used to quantify the size of cardiomyocytes that were round and contained a nucleus. At least 500 cells per sample were quantified.

KI-HDR using ssODN template insertion. Mice (BalbcJ, JAX stock 000651) harboring the mutated Tnnt2 alleles were generated using CRISPR/Cas9 reagents. The guide RNAs and donor ssODN were designed to delete Lysine residue K210 and introduce a silent Clal restriction site for genotyping The was purposes. sgRNA sequence AGAAGAAGATCCTGGCAGAG (SEQ ID NO: 5). The guide was selected using the CRISPR Design Tool (http://tools.genome-engineering.org). crRNA and tracRNA were annealed and mixed with Cas9 protein to form a ribonucleotide protein complex. The ssODN (IDT) [AGACAGAGCGGAAGAGTGGGAAGAGACAGACAGAGAGAGAGAAGAAGAAAATCCTGGCC GAGCGAAGGAAGGCGCTGGCAATCGATCATCTGAATGAAGACCAACTGAGGTGGGGACA GTTGGTTGGGTGGCCCCTGGCACTCTTCCTGA (SEQ ID NO: 6)] was added to the mix and the cocktail was microinjected into the cytoplasm of fertilized one-cell eggs isolated from super ovulated females. The eggs were incubated in media containing cytochalasin-B immediately before and during microinjection to improve egg survival. The surviving eggs were transferred into the oviducts of day 0.5 pseudo pregnant recipient ICR females (Envigo, Inc.) to produce putative founder mice. Founder mice were identified via PCR using the primer set 5′-TGGGTCTTTCTCTCATGGTTTCC-3′ (SEQ ID NO: 7) and 5′-GCTCAGATAAGAAAAGGGCCT-3′ (SEQ ID NO: 8) and the amplicon was submitted for Sanger sequencing. F0 mice were bred with BalbcJ mice to obtain F1 mice heterozygous for the mutated allele.

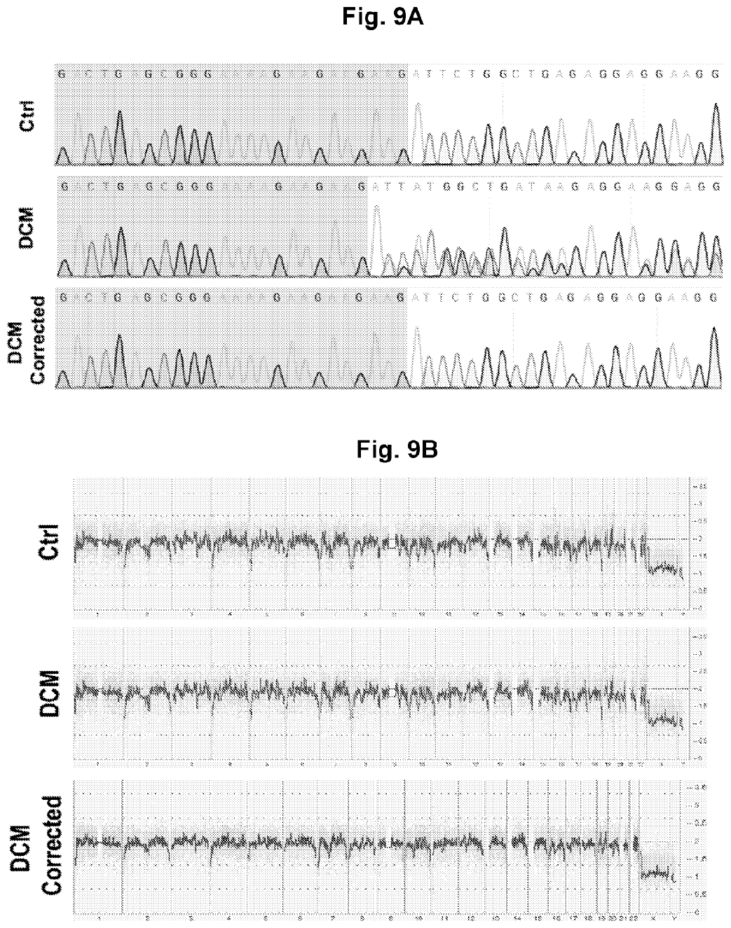

In each experiment, the age of the mouse is indicated in the text and the figure. Littermate controls were used whenever possible. No statistical methods were used to predetermine sample size. All echocardiographic studies were carried out blinded to the treatment of the mice during the experiments and outcome assessments (FIGS. 9A-9B).

Statistical analysis. Data were analyzed and graphed using Prism (GraphPad). Data were presented as mean±SEM. Comparisons were conducted via either two-tailed Student's t-test or one-way ANOVA with statistically significant differences defined by p<0.05 (*), p<0.01 (**), p<0.001 (***), and p<0.0001 (****).

What is claimed is:

1. A method of improving heart function in a subject in need thereof comprising administering at least one bisphosphonate, pharmaceutically acceptable salt thereof, or any hydrate thereof,
wherein the subject in need thereof has or is suspected of having dilated cardiomyopathy (DCM).

2. The method of claim 1, wherein the at least one bisphosphonate, pharmaceutically acceptable salt thereof, or any hydrate thereof comprises risedronate.

3. The method of either claim 1 or claim 2, wherein the subject in need thereof has or is suspected of having familial DCM.

4. The method of any one of claims 1-3, wherein the subject in need thereof has or is suspected of having a deletion mutation at lysine 210 (K210) in a cardiac troponin T gene.

5. The method of any one of claims 1-4, wherein the at least one bisphosphonate, pharmaceutically acceptable salt thereof, or any hydrate thereof is administered parenterally.

6. The method of any one of claims 1-5, wherein the at least one bisphosphonate, pharmaceutically acceptable salt thereof, or any hydrate thereof is administered intravenously, subcutaneously, intramuscularly, transdermally, or any combination thereof.

7. The method of any one of claims 1-6, wherein administering the at least one bisphosphonate, pharmaceutically acceptable salt thereof, or any hydrate thereof improves heart function in the subject in need thereof compared to an untreated subject with identical disease condition and predicted outcome.

8. The method of any one of claims 1-7, wherein administering the at least one bisphosphonate, pharmaceutically acceptable salt thereof, or any hydrate thereof increases ejection fraction in the subject in need thereof compared to an untreated subject with identical disease condition and predicted outcome.

9. The method of any one of claims 1-8, wherein administering the at least one bisphosphonate, pharmaceutically acceptable salt thereof, or any hydrate thereof decreases cardiac hypertrophy compared to an untreated subject with identical disease condition and predicted outcome.

10. A method of treating or preventing dilated cardiomyopathy (DCM) in a subject comprising administering to a subject having or suspected of having DCM an effective amount of at least one bisphosphonate, pharmaceutically acceptable salt thereof, or any hydrate thereof,
wherein the effective amount of the at least one bisphosphonate, pharmaceutically acceptable salt thereof, or any hydrate thereof comprises an amount that improves at least one characteristic of DCM.

11. The method of claim 10, wherein the at least one characteristic of DCM comprises ventricular dilation, systolic dysfunction, or both.

12. The method of either claim 10 or claim 11, wherein the at least one bisphosphonate, pharmaceutically acceptable salt thereof, or any hydrate thereof comprises risedronate.

13. The method of any one of claims 10-12, wherein the subject having or suspected of having DCM has or is suspected of having familial DCM.

14. The method of claim 13, wherein the subject having or suspected of having familial DCM has a deletion mutation at lysine 210 (K210) in a cardiac troponin T gene.

15. The method of any one of claims 10-14, wherein the at least one bisphosphonate, pharmaceutically acceptable salt thereof, or any hydrate thereof is administered parenterally.

16. The method of any one of claims 10-15, wherein the at least one bisphosphonate, pharmaceutically acceptable salt thereof, or any hydrate thereof is administered intravenously, subcutaneously, intramuscularly, transdermally, or any combination thereof.

17. The method of any one of claims 10-16, wherein administering at least one bisphosphonate, pharmaceutically acceptable salt thereof, or any hydrate thereof increases fractional shortening, ejection fraction, stroke volume, cardiac output, or any combination thereof in the subject compared to an untreated subject with identical disease condition and predicted outcome.

18. The method of any one of claims 10-17, further comprising administering to the subject at least one agent to manage one or more symptoms associated with DCM.

19. The method of claim 18, wherein the at least one agent comprises an angiotensin-converting enzyme (ACE) inhibitor, an angiotensin receptor blocker (ARB), a beta-blocker, an aldosterone receptor antagonist, a neural endopeptidase inhibitor, a diuretic, a mineralocorticoid receptor blocker, or any combination thereof.

20. The method of claim 18, wherein the one or more symptoms associated with DCM comprise fatigue, dyspnea, edema, heart palpitations, heart murmurs, or any combination thereof.
